SEQUENCE LISTING (15 sequences)

SEQ ID NO: 1 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 2 - protein, 154 aa, Petromyzon marinus
KQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNE

SEQ ID NO: 3 - protein, 331 aa, Petromyzon marinus
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGP

SEQ ID NO: 4 - protein, 130 aa, Petromyzon marinus
VRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSE

SEQ ID NO: 5 - protein, 219 aa, Petromyzon marinus
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 6 - protein, 331 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGPSSIPNPLLGLD

SEQ ID NO: 7 - protein, 123 aa, Artificial Sequence
  Sequence created by mutation selection
QVQLVESGGRLVQAGGSLRVACVASGGTFSSYVMGWFRQAPGKERESVASITRDGDTTFYADSVKGRFTIARDNAKNTVYLQMNSLQPEDTGVYYCAAAVQYGTLWSRQYDYWGQGTQVTVSS

SEQ ID NO: 8 - protein, 263 aa, Artificial Sequence
  Created by clone selection; 1-123 and 141-end define a humanized VHH
QVQLVESGGGLVQPGGSLRLSCAASGGTFSSYVMGWFRQAPGKGLESVASITRDGDTTFYADSVKGRFTISRDNSKNTLYLQMNSLRAEDTAVYYCAAAVQYGTLWSRQYDYWGQGTLVTVSSAHHSEDPSSKAPKAPMAQVQLVESGGGLVQPGGSLRLSCAASGGTFSSYVMGWFRQAPGKGLESVASITRDGDTTFYADSVKGRFTISRDNSKNTLYLQMNSLRAEDTAVYYCAAAVQYGTLWSRQYDYWGQGTLVTVSS

SEQ ID NO: 9 - protein, 348 aa, Artificial Sequence
  Clone selection and creation of sea lamprey-human chimera. AAs 228-239 are from ERDBD
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSSGSGGGGSGGGGSGGGGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDIPNPLLGLD

SEQ ID NO: 10 - protein, 114 aa, Oryctolagus cuniculus
MAVESAKETRYCAVCNDYASGYHYGVWSCEGCKAFFKRSIQGNRHNDYMCPATNQCTIDKNRRKSCQACRLRKCYEVGMMKGGIRKDRRGGRMLKHKRQRDDGEGRNETGPPGD

SEQ ID NO: 11 - protein, 110 aa, Gallus gallus
LSMESTKETRYCAVCNDYASGYHYGVWSCEGCKAFFKRSIQGHNDYMCPATNQCTIDKNRRKSCQACRLRKCYEVGMMKGGIRKDRRGGRMMKQKRQREEQDSRNGEASS

SEQ ID NO: 12 - protein, 100 aa, Gallus gallus
SKRDAHFCAVCSDYASGYHYGVWSCEGCKAFFKRSIQGHNDYICPATNQCTIDKNRRKSCQACRLRKCYEVGMMKCGSRRERCGYRILRRHRNSEDCMGK

SEQ ID NO: 13 - protein, 387 aa, Artificial Sequence
  sea lamprey-yeast chimera. AAs 228-378 are GalDBD
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAVSSGSGGGGSGGGGSGGGGSMAMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSMAIPNPLLGLD

SEQ ID NO: 14 - protein, 112 aa, homo sapiens
MAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGD

SEQ ID NO: 15 - protein, 17 aa, Lama glama
AHHSEDPSSKAPKAPMA

(19) United States
(12) Patent Application Publication
(10) Pub. No.: US 2017/0183645 A1
(43) Pub. Date: Jun. 29, 2017

(54) COMPOSITION AND METHOD FOR DIVERSIFYING POLYPEPTIDE LIBRARIES

(71) Applicant: Abzyme Therapeutics LLC., Pottstown, PA (US)

(72) Inventors: Hiep Tran, West Chester, PA (US); Alexander Taylor, Mount Juliet, TN (US); Gisela Marquez, Exton, PA (US); Christine Prokopowitz, Eagleville, PA (US); Rolf Swoboda, Upper Darby, PA (US)

(21) Appl. No.: 15/380,105

(22) Filed: Dec. 15, 2016

Related U.S. Application Data
(60) Provisional application No. 62/387,511, filed on Dec. 24, 2015.

(51) Int. Cl. C12N 15/10; C07K 16/00; C12N 15/81; G01N 33/569; C12N 9/78

(52) U.S. Cl. CPC C12N 15/1037; G01N 33/56961; C12N 9/78; C12Y 305/04005; C12N 15/81; C07K 16/00; C07K 2317/14; C07K 2317/22; C07K 2317/569

(57) ABSTRACT

Provided, among other things, is a yeast cell comprising: (A) a recombinant DNA that constitutively or inducibly expresses a cytidine deaminase comprising sequence with about 90% sequence identity or more with a cytidine deaminase domain of (i) SEQ ID NO. 2 or SEQ ID NO. 4, or (ii) a chimera between the two starting with SEQ ID NO. 3 or SEQ ID NO. 4 sequence and having one transition to end in SEQ ID NO. 1 or SEQ ID NO. 2 sequence, or (iii) a chimera between the two starting with SEQ ID NO. 1 or SEQ ID NO. 2 sequence and having one transition to end in SEQ ID NO. 3 or SEQ ID NO. 4 sequence; and (B) a second recombinant DNA that constitutively or inducibly expresses a binding scaffold protein for presentation on the outer surface of the yeast, wherein the cytidine deaminase as expressed by the first recombinant DNA is effective to contribute to a mutagenic process for inducing mutations in the binding scaffold protein of the yeast cell.

### CROSS-REFERENCE TO RELATED APPLICATION

This application claims benefit of U.S. Provisional Patent Application No. 62/387,511 filed Dec. 24, 2015, which is incorporated herein by reference in its entirety.

### SEQUENCE LISTING STATEMENT

Filed herewith is a Sequence Listing (name: ABZ001SeqListing_ST25.txt; created: Dec. 13, 2016; sized: 27 KB). The content of that Sequence Listing is incorporated herein by reference in its entirety.

### FIELD OF THE INVENTION

The present application relates generally to a novel diversification and maturation method for polypeptide libraries using a yeast-based expression system. More specifically, the yeast system disclosed facilitates the polypeptide library diversification, protein maturation and screening of binder proteins with modified affinity to another molecule.

Antibodies have been widely accepted for treatment of a variety of diseases, including cancer, arthritis and infectious diseases. Currently more than 300 monoclonal antibody-based drugs are in clinical trials. The predominant advantage of antibody-mediated therapy is its high specificity, facilitated by direct binding to the target(s) for neutralization or elimination (KRAEBER-BODERE et al. 2014). As of Nov. 10, 2014, forty-seven monoclonal antibody products have been approved for medical use in the US or Europe (ECKER et al. 2015). Nevertheless development of antibodies as research reagents and therapeutics with high affinity and specificity remains both time-consuming and labor-intensive. Accordingly an animal-free, high throughput platform for antibody discovery and isolation would accelerate the antibody generation process.

Success in generation of highly specific antibodies in an ex-vivo system depends on the ability to establish highly diverse heavy and light chain libraries together with efficient screening capacity. Currently ex-vivo non-mammalian approaches for generating antibodies such as phage display (HAWKINS et al. 1992), yeast surface display (BODER and WITTRUP 1997; BODER and WITTRUP 2000), ribosome display (HANES and PLUCKTHUN 1997; HE and TAUSSIG 1997), RNA display (REIERSEN et al. 2005), and mammalian cell display (BEERLI et al. 2008) are not intrinsically capable of affinity maturation because they lack the capacity to effect somatic hypermutation. Alternatively, error-prone-PCR followed by labor-intensive sub-library re-cloning steps are generally incorporated into all current ex-vivo systems to generate high-affinity antibodies (CHAO et al. 2006) and U.S. Pat. No. 8,691,730. This method is easily doable if the antibody is expressed by a single gene such as in the single-chain variable fragment (scFv) format. When antibodies consist of separate light and heavy chain genes, error-prone PCR sub-libraries have to be constructed for each antigen-specific clone to maintain the correct heavy-light chain pairing. Otherwise random pairing of a light chain from one active antibody with a heavy chain from a different clone will not likely generate again a target-specific antibody.

In both prokaryotic and eukaryotic cells, there is a complex system that maintains genomic integrity, including e.g., the DNA polymerase complex copies DNA with high fidelity; the post-replication mismatch repair system repairs errors generated during replication; the DNA recombination machinery repairs DNA damage via homologous recombination, and the excision repair pathway corrects DNA adducts (BURGERS 1998; HOUSE et al. 2014; KOLODNER and MARSISCHKY 1999; NISHINO and MORIKAWA 2002). Nevertheless, there are a number of approaches that can be used to induce mutation in cells for effecting antibody diversification. Inactivation of the mismatch pathway has been shown to cause hypermutation (MARTI et al. 2002). Overexpression of dominant negative alleles of mismatch repair genes to impair the cell mismatch repair pathway ((DROTSCHMANN et al. 1999), U.S. Pat. Nos. 6,808,894 and 6,921,666) leads to the mutator phenotype. Chemicals also can be used to inhibit the mismatch repair pathway. For example, the mutagenic effect of cadmium is caused by its inhibition of the mismatch repair pathway JIN et al. 2003). Inactivation of the proof-reading activity of DNA polymerases such as mutation in Pol2 exonuclease (MORRISON et al. 1991), the DNA recombination pathway such as Rad52 deletion (ENDO et al. 2007) or the excision repair pathway such as Rad3 deletion (MONTELONE et al. 1992) also cause mutator phenotypes. Exposure of cells to DNA damaging radiation or chemical agents such as UV irradiation or MMS also can elevate mutation rates (MONTELONE et al. 1981). It is noted that the approaches mentioned above cause global and unspecific DNA damage or errors that limits the usage for antibody maturation. For example, inactivation of the mismatch repair pathway causes mutations primarily in homonucleotide runs (TRAN et al. 1997) resulting in frameshift mutations of open reading frames which are not suitable for antibody gene maturation.

For antibody maturation, it is preferred to have hypermutation caused by base substitutions as the latter do not cause shifts of the open reading frame. Substitution mutations in general are generated during DNA replication by nucleotide mis-incorporation either caused by infidelity of DNA polymerases, or by the presence of ambiguous nucleotide analogues (e.g. the nucleotide analog 6N-hydroxylamino purine can pair with either cytidine or thymine), or modification of the DNA template (e.g. Cytosine is deaminated to Uracil that in turn mimics Thymine pairing with Adenine). Cytosine-to-Uracil deamination occurs either spontaneously or by the action of cytosine deaminases (DUNCAN and MILLER 1980)(AKA cytidine deaminases). Normally, cytosine to uracil deamination products are excised from DNA by the enzyme uracil-DNA glycosylase. When uracil-DNA glycosylase (Ung−) is lacking, the deamination of cytosine becomes a significant source of mutations (DUNCAN and MILLER 1980). It has been shown that overexpression of cytidine deaminases in combination with inactivation of uracil-DNA glycosylase results in synergistic mutator effects (MAYOROV et al. 2005a).

In mammals, the action of activation-induced deaminase (AID) is essential for functional antigen receptor maturation, by mediating class switching, gene conversion and hypermutation (for review see (KATO et al. 2012)). Muramatsu and co-workers (MURAMATSU et al. 2000) have shown that AID^{-/-} spleen cells stimulated in vitro with LPS and certain cytokines failed to undergo class switch recombination and immunization of AID^{-/-} mice with 4-hydroxy-3-nitrophenylacetyl (NP) failed to accumulate mutations in the NP-specific variable region gene or class switching. Mammalian AID, cloned and isolated by Muramatsu and coworkers (MURAMATSU et al. 1999) has been shown to have cytidine deaminase activity. Therefore, it was not surprising that overexpression of mammalian activation-induced cytidine deaminase (AID) (PETERSEN-MAHRT et al. 2002) and its homologs APOBEC1, APOBEC3C and APOBEC3G (HARRIS et al. 2002) is mutagenic in E. coli Ung− mutant strains

Based on the mutator effect resulting from overexpression of human Activation-induced cytidine deaminase (AID) (PETERSEN-MAHRT et al. 2002) and its mammalian homologs APOBEC1, APOBEC3C and APOBEC3G (HARRIS et al. 2002) in E. coli, U.S. Pat. No. 7,820,442 (2010) and U.S. Pat. No. 8,288,160 (2012) entitled “Activation Induced Deaminase (AID)” were issued to these authors, and claimed the use of mammalian AID and its homologs, selected from the group consisting of Apobec-1, Apobec3C, and Apobec3G, in antibody diversification in both prokaryotic and eukaryotic cells.

Activation-induced cytidine deaminase (AID) and APOBEC (“apolipoprotein B mRNA editing enzyme, catalytic polypeptide-like”) are members of the large and diverse cytidine deaminase family. Cytidine deaminases have been reported in various organisms including bacteria, yeast, tetrapods, jawless vertebrates and jawed vertebrates (ROGOZIN et al. 2007). Interestingly, because of the diversity of the cytidine deaminase family, overexpression of different cytidine deaminases in general or AID/APOBEC-like in particular in yeast and bacteria may or may not result in mutagenic phenotype or may have different mutagenic specificities (LADA et al. 2011). The expression of yeast CDD1 and TAD2/TAD3, human APOBEC2 and APOBEC4, Xanthomonas oryzae APOBEC5, and deaminase encoded by Micromonas sp. gene MICPUN_56782 was nonmutagenic (LADA et al. 2011). While human AID, rat APOBEC1, lamprey CDA1, and human APOBEC3G enzymes cause mutations when overproduced ectopically in bacteria or yeast, their mutagenic specificities are strikingly different (LADA et al. 2011). Thus to optimally diversify antibody or polypetide-encoding genes there is need for selecting the most efficient cytidine deaminase, expressing it in a defined genetic host system and growth conditions thereby effectively achieving the desired protein diversification.

The invention provided is a method and composition consisting of a highly engineered yeast mutator strain, expressing a jawless vertebrate sea lamprey cytidine deaminase as an active mutator gene, and grown in the presence of a mutation-inducing chemical, that together are used to diversify a library of polypeptides, that are cell surface accessible for binding to macromolecules. Jawless vertebrates have a fundamentally different immunological system compared to that of jawed vertebrates. Instead of immunoglobulins, the variable lymphocyte receptors (VLRs) of jawless fish are composed of highly diverse leucine-rich repeats (LRRs) (for review see (ROGOZIN et al. 2007)). In jawed vertebrates, maturation of immunoglobulins occurs by RAG1/RAG2-mediated V(D)J recombination, AID-induced class switching and hypermutation (for review see (ROGOZIN et al. 2007)). Jawless vertebrates use different strategies to generate highly diverse VLRs that are based on RAG-independent combinatorial assembly of leucine-rich repeats (PANCER et al. 2004). The VLR gene assembly mechanism appears to be of the gene conversion type because of the nonreciprocal insertion of LRR cassettes into the intervening sequences of the germ line gene (KATO et al. 2012; ROGOZIN et al. 2007). The gene conversion is mediated by cytidine deaminase 1 (CDA1) and cytidine deaminase 2 (CDA2) which have been identified in jawless fish sea lamprey Petromyzon marinus (KATO et al. 2012; ROGOZIN et al. 2007). Unlike AID, the PmCDAs are expressed constitutively, manifested by the fact that the levels and pattern of PmCDAs expression do not change in response to stimulation (KATO et al. 2012); PmCDAs therefore are not activation-induced cytidine deaminases in contrast to mammalian AIDs. Hence it appears that PmCDA does not mediate somatic hypermution in vivo, but rather mediates somatic diversification via a gene conversion mechanism similar to rabbits and chickens. Hence based on its in vivo role it is not obvious that PmCDA could be useful for in vitro affinity maturation. Moreover it is therefore not a direct functional homolog of AID.

The expression of CDA1 in E. coli and yeast causes strong mutator phenotypes (ROGOZIN et al. 2007). In fact, compared to the expression of human AID, human APOBEC3G and rat APOBEC1, the expression of lamprey CDA1 produce the strongest mutator phenotype in both UNG⁺ and ung− yeast strains as measured with both CAN1 forward mutations and TAG and TAA nonsense reversions (LADA et al. 2011). In contrast, there is no report describing a mutator effect of sea lamprey CDA2 when it is expressed in bacteria and yeast. As DNA encoding leucine-rich repeat VLRs are CDA intrinsic substrates and there are no immunoglobulins in jawless vertebrates, the mutator effect of the PmCDA expression in yeast on genes encoding immunoglobulins as well as other scaffolds has been unknown.

There is a continuing need in the art for improving the generation of binding-specific antibodies or antibody analogs. This invention is a novel technology platform, called Self-Diversifying Antibody Library or SDALib that comprises a monoclonal antibody generating system providing a diverse array of complete antibodies in vitro, without using in vivo immunization. The invented system can be used for polypeptide library diversification, protein maturation and screening of binder proteins with modified affinity to another molecule. Advantages of this invention can include a low cost, rapid growth eukaryotic protein expression and surface display system with ease of culture and culture maintenance, facile manipulation and genetic engineering. Moreover, yeast mating allows random combination of antibody heavy chain and light chain libraries to form a combined library with highly diverse random H/L combinations. The expression of lamprey CDA—the most powerful deaminase mutator of DNA in yeast in combination with the chemical supermutagen HAP allows rapid library diversification. Finally the use of diploid and/or polyploid yeast strains versus the normally used haploid yeast version protect yeast cells from lethal mutation damage due to the presence of two or more copies of essential genes. In combination with panning and Fluorescence Assisted Cell Sorting (FACS) yeast cells expressing functional binders can be quickly identified and isolated.

### SUMMARY OF THE INVENTION

The present invention provides a method and kit for diversifying a polypeptide library and selecting binders to a target of interest. In a preferred embodiment, methods and kits for isolating camelid single domain VHH antibodies are disclosed. In other embodiments, the cell-based self-diversifying methods are used to isolate human heavy-chain only single domain antibodies, human single chain variable fragments (ScFv) and human traditional antibodies. The cell-based self-diversifying platform has additional applications in diversifying other binders and maturating binders to modulate their functional activity.

In an exemplary embodiment of the invention, influenza H5N1 neuraminidase is presented as an antigen of interest. Genetically engineered host cells comprising a self-diversifying surface display camelid VHH antibody library are then contacted with the antigen target. The engineered cells expressing antibodies reactive to the antigen is enriched by biological panning and isolated by Fluorescence-Activated Cell Sorting or FACS. Antibodies from sorted cells are purified and confirmed for the target-specific binding activity.

In another embodiment of the invention, a cell-based system for protein binder discovery is provided, wherein the system comprises a) a first DNA construct comprising a nucleic acid molecule encoding a protein scaffold operably linked to a promoter and a DNA motif; b) a second DNA construct having a nucleic acid molecule encoding a second polypeptide operably linked to a promoter; c) diversifying cell culture media supplemented with protein-expression inducer and mutation-causing chemicals; and d) Two yeast strains of opposite mating types; the first yeast strain contains said first DNA construct; the second yeast strain contains said second construct; the final host cell formed by mating the first and the second yeast strains comprising said first and second DNA constructs, diversification of said protein scaffold in said host cell being dependent upon enzymatic activity of said second polypeptide or the presence of the mutation-causing chemicals supplemented in said cell culture media.

In yet another embodiment, a method for isolating binders to a target of interest with modulated binding activity is provided, wherein the method comprises: a) providing a host cell containing a first DNA construct comprising a nucleic acid molecule encoding a protein scaffold operably linked to a first promoter and a DNA recognition sequence; a second DNA construct having a nucleic acid molecule encoding a second polypeptide operably linked to a second promoter; and diversifying cell culture media supplemented with protein-expression inducers and mutation-causing chemicals; b) culturing said host cells in said diversifying media to diversify the said binder encoding genes; and c) isolating host cell expressing scaffold reactive to a target by either biological panning or FACS.

In a particular embodiment, the first DNA construct encodes a scaffold selected from the group of immunoglobulin heavy chain or light chain variable regions or polypeptide scaffolds including, but not limited to Anticalins, fibronectin type III domain—Adnectins, Designed Ankyrin Repeat Protein or DARPins and Centyrins. In a particular embodiment, the second DNA construct encodes cytidine deaminases selected from group of sea lamprey cytidine deaminase 1 (PmCDA1), chimeric cytidine deaminase CDA2/CDA1 and their variants.

### DESCRIPTION OF THE DRAWINGS AND SEQUENCE LISTING

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only illustrative embodiments of this invention and are therefore not to be considered limiting of its scope, for the invention may admit to other equally effective embodiments.

FIGS. 1A and 1B show a schematic diagrams of yeast expression plasmids utilized in the practice of the present invention. FIG. 1A—replicative multicopy plasmid. FIG. 1B—integrative plasmid obtained from plasmid presented in FIG. 1A by deleting SnaBI-BsmBI region of the yeast 2-micron replication origin. A yeast expression plasmid is shown which encodes full-length lamprey cytidine deaminase 1. These yeast-E. coli shuttle plasmids contain LEU2 as a yeast transformation marker.

FIG. 1C shows a sequence alignment of sea lamprey CDA1 (PmCDA1, SEQ ID NO. 1) sea lamprey CDA2 (PmCDA2, SEQ ID NO. 3), chimeras between the two CDA1 and CDA2 (SEQ ID NO. 5 and SEQ ID NO. 6).

FIG. 1D is an illustration of the CDA1 and CDA2 sequences showing the domains that are cytidine deaminase-like (20-173 of SEQ ID NO. 1, designated SEQ ID NO. 2; 15-144 of SEQ ID NO. 3, designated SEQ ID NO. 4). These are believed to be the domains that carry cytidine deaminase activity.

FIGS. 2A to 2E show vectors for expressing the first polypeptide constructs. FIG. 2A is representative of one camelid VHH cloned in frame with yeast invertase secretory signal at the N-terminus and membrane anchor AGA2 at C-terminus. A gene encoding a camelid heavy chain single domain variable was cloned directionally into SfiI-SfiI sites. The fusion construct is expressed under a galactose inducible promoter, Gal1/10. This yeast-E. coli centromeric shuttle plasmid contains TRP1 as a yeast transformation marker. FIG. 2B represents a backbone vector for expression of human heavy chain variable domains where human VH can be cloned at EagI-SalI sites in frame with yeast invertase secretory signal at the N-terminus and membrane anchor AGA2 at C-terminus. When the first construct is a heterodimeric protein such as an antibody Fab fragment two backbone vectors are used for co-expression—one vector (FIG. 2C) with TRP1 selection marker is for expressing antibody heavy chain; the heterodimeric partner, antibody light chain lambda or light chain kappa is expressed in vectors with HIS3 selection marker presented in FIG. 2D and FIG. 2E, respectively.

FIGS. 3A-3C show a hypermutation patch test FIG. 3A, FIG. 3C and a spot test FIG. 3B of forward mutations in the yeast CAN1 gene induced by overexpression of full length cytidine deaminase PmCDA1 (FIG. 3A), various variants of PmCDA1 and PmCDA2 (FIG. 3C) and exposure to the replication fidelity compromising compound HAP (FIG. 3B). Left sides—no inducers are present; Right side—yeast are exposed to inducers.

FIG. 4 is a schematic diagram of the antibody discovery system of the present invention.

FIG. 5 shows graphs showing that the diversification and panning system of the invention efficiently sorts a cell population expressing camelid antibodies with high affinity binding to H5N1 neuraminidase target. Enriched library is labeled with anti-FLAG Dylight649 and NA-biotin/streptavidin-FITC to monitor the VHH display efficiency and activity to NA. The double-positive sorted cell fraction is indicated.

FIG. 6 is a graph showing an alignment tree of nineteen anti-N1 NA VHH antibodies isolated using SDALib platform. Amino acid sequence of representative VHH clone Flu 27-8 is shown in SEQ ID 7.

FIG. 7 is a graph showing camelid VHH expressed and purified from a bacterial system. SDS-PAGE of E. coli produced N1-NA monovalent (lanes 1, 2 and 3) and bivalent (lane 4) camelid antibodies.

The Sequence Listing filed with this application as a text file includes the following protein sequences:

| | |
|---|---|
| SEQ ID NO. 1 | Sea lamprey CDA1 |
| SEQ ID NO. 2 | Sea lamprey CDA1 Cytindine deaminase-like domain |
| SEQ ID NO. 3 | Sea lamprey CDA2 |
| SEQ ID NO. 4 | Sea lamprey CDA2 Cytindine deaminase-like doma |
| SEQ ID NO. 5 | Sea lamprey CDA2-CDA1 hybrid (2/1 hybrid) |
| SEQ ID NO. 6 | Sea lamprey CDA1-CDA2 hybrid (1/2 hybrid) |
| SEQ ID NO. 7 | Anti-Flu neuraminidase VHH (clone Flu 27-8) |
| SEQ ID NO. 8 | Bivalent homodimer of humanized anti-Flu |
| | neuraminidase VHH (clone Flu h27-8GL.h27-8GL). |
| | AAs 1-123 and 141-262 define a humanized camelid |
| | VHH |
| SEQ ID NO. 9 | Sea lamprey CDA1 fused with human estrogen |
| | receptor DNA binding domain (ERDBD) and V5 |
| | epitope. AAs 228-239 represent human ERDBD |
| SEQ ID NO. 10 | Rabbit ERDBD (94% Sequence ID to human) |
| SEQ ID NO. 11 | Chicken alpha ERDBD (93% Sequence ID to human) |
| SEQ ID NO. 12 | Chicken beta ERDBD (87% Sequence ID to human) |
| SEQ ID NO. 13 | Sea lamprey CDA1 fused with yeast Gal4 DNA |
| | binding domain (Gal4DBD) and V5 epitope. AAs |
| | 228-378 are Gal4DBD |
| SEQ ID NO. 14 | Human ERDBD |
| SEQ ID NO. 15 | Structural upper hinge of the llama IgG2 |

To facilitate understanding, identical reference numerals have been used, where possible, to designate comparable elements that are common to the figures. The figures are not drawn to scale and may be simplified for clarity. It is contemplated that elements and features of one embodiment may be beneficially incorporated in other embodiments without further recitation.

### DETAILED DESCRIPTION

In accordance with the present invention, compositions, methods and kits are provided for polypeptide diversification and isolation of a binder protein to a target of interest. A yeast-based system is disclosed to produce protein scaffolds reactive to a target. In a preferred aspect of the invention, an antibody discovery method is provided which enables isolating target-specific antibodies starting from a naïve antibody library.

### I. Definitions

The following definitions are provided to facilitate an understanding of the present invention.

A “promoter” is a DNA sequence located proximal to the start of transcription at the 5′ end of an operably linked transcribed sequence. The promoter can contain one or more regulatory elements or modules that act together in coordinating and regulating transcription of the operably linked gene. An inducible promoter is a promoter that responds to the presence of different biochemical stimuli. Such promoters include, but are not limited, to the CUP1 promoter, heat shock promoters, galactose-inducible promoters, glycolytic promoters such as alcohol dehydrogenase (ADH) glyceraldehyde phosphate dehydrogenase (GPD) and the like.

“Operably linked” describes two macromolecular elements arranged such that modulating the activity of the first element induces an effect on the second element. In this manner, modulation of the activity of a promoter element can be used to alter and/or regulate the expression of an operably linked coding sequence. For example, the transcription of a coding sequence that is operably linked to a promoter element is induced by factors that “activate” the promoter's activity; transcription of a coding sequence that is operably-linked to a promoter element is inhibited by factors that “repress” the promoter's activity. Thus, a promoter region is operably linked to the coding sequence of a protein if transcription of such coding sequence activity is influenced by the activity of the promoter.

“Fusion construct” refers generally to recombinant genes which encode fusion proteins. Such fusion constructs can include operably linked nucleic acids isolated from two or more different genes.

A “fusion protein” is a hybrid protein, i.e., a protein that has been constructed to contain domains from at least two different proteins. An exemplary fusion protein, as described herein is a hybrid protein which possesses (a) a transcriptional regulatory domain from a transcriptional regulatory protein, or (b) a DNA binding domain from a DNA binding protein linked to a heterologous protein to be assayed for interaction. The structure of the fusion protein is such that the transcriptional regulatory domain and the DNA binding domain are arranged in a manner that allows both domains to be biologically active. The protein that is the source of the transcriptional regulatory domain is different from the protein that is the source of the DNA binding domain. In other words, the two domains are heterologous to each other. The transcriptional regulatory domain of the fusion protein can either activate or repress transcription of target genes, depending on the native biological activity of the domain.

The term “fusion protein gene” refers to a DNA sequence that encodes a fusion protein. A fusion protein gene can further provide transcriptional and translational regulatory elements for the transcriptional and translational control thereof.

A nucleic acid molecule, such as a DNA or gene is said to be “capable of expressing” a polypeptide if the molecule contains the coding sequences for the polypeptide operably linked to expression control sequences (e.g., promoter sequence) which, in the appropriate host environment, facilitate transcription, processing and translation of the encoded genetic information into a protein product.

The phrase “consisting essentially of” when referring to a particular nucleotide or amino acid means a sequence having the properties of a given reference sequence. For example, when used in reference to an amino acid sequence, the phrase includes the sequence per se and molecular modifications that would not affect the basic and novel characteristics of the sequence.

As used herein, a “cloning vector” is any entity that is capable of delivering a nucleic acid sequence into a host cell for cloning purposes. Examples of cloning vectors include plasmids or phage genomes. A plasmid which replicates autonomously in a host cell is especially preferred. Alternatively, a nucleic acid molecule which stably integrates into the host cell's chromosomal DNA and is inherited by daughter cells can be employed. Optionally, such vectors include a number of endonuclease recognition sites to facilitate manipulation of the sequence in a controlled and targeted fashion. Cloning vectors of the invention can also comprise sequences conferring resistance to selection agents, often referred to herein as selectable marker genes. For example, “a marker gene” can be a gene which confers resistance to a specific antibiotic on a host cell.

As used herein, an “expression vector” is a vehicle or vector similar to the cloning vector but is especially designed to provide an environment that facilitates expression of the cloned gene product after transformation into the host. Such vectors contain regulatory elements for expression in prokaryotic and/or eukaryotic hosts as well as sequences conferring selection properties of cells containing the expression vector. Optionally, enhancer elements, termination sequences, tissue-specificity elements, and/or translational initiation and termination sites can be included.

A “host” refers to any organism or cell line that is the recipient of a cloning or expression vector. In preferred embodiments, the host of the invention is a yeast cell or a cultured animal cell such as a mammalian or insect cell. Especially preferred is the yeast host Saccharomyces cerevisiae.

A “transformed cell” is any cell into which (or into an ancestor of which) exogenous DNA has been introduced by means of recombinant DNA techniques or cell fusion, e.g. mating.

“Response elements” are specific DNA sequences located in promoters of inducible genes; such inducers can include chemicals, hormones, metals such as zinc, cadmium or copper, temperature changes (e.g. heat shock) and transcription factors. In many instances, nuclear receptors in the form of homodimers, heterodimers or monomers bind specifically to DNA response elements to activate or repress transcription of the targeted genes in the presence or the absence of ligands for the nuclear receptors.

The terms “variant” or “derivative” in relation to lamprey CDA1 polypeptide includes any substitution of, variation of, modification of, replacement of, deletion of or addition of one (or more) amino acids from or to the polypeptide sequence of CDA1. Preferably, nucleic acids encoding CDA1 are understood to comprise variants or derivatives thereof.

Such “modifications” of CDA1 polypeptides include fusion proteins in which CDA1 polypeptide or a portion or fragment thereof is linked to or fused to another polypeptide or molecule.

The term “homologue” as used herein with respect to the nucleotide sequence and the amino acid sequence of CDA1 can be synonymous with allelic variations in the CDA1 sequences and includes known homologues.

The “functional activity” of a protein in the context of the present invention describes the function the protein performs in its tested environment. Altering or modulating the functional activity of a protein includes within its scope increasing, decreasing or otherwise altering the native activity of the protein itself. In addition, it also includes within its scope increasing or decreasing the level of expression and/or altering the intracellular distribution of the nucleic acid encoding the protein, and/or altering the intracellular distribution of the protein itself. By “cytidine deaminase mutation activity” or “mutator activity” is meant the functional activity of cytidine deaminase or its homologues to increase mutation above background without the presence of the enzyme.

The term “expression” refers to the transcription of a gene's DNA template to produce the corresponding mRNA and translation of this mRNA to produce the corresponding gene product (i.e., a peptide, polypeptide, or protein). The tem “activates gene expression” refers to inducing or increasing the transcription of a gene in response to a treatment where such induction or increase is compared to the amount of gene expression in the absence of said treatment. Similarly, the terms “decreases gene expression” or “down-regulates gene expression” refers to inhibiting or blocking the transcription of a gene in response to a treatment and where such decrease or down-regulation is compared to the amount of gene expression in the absence of said treatment.

The “mutation rate” is the rate at which a particular mutation occurs, usually given as the number of events per gene per generation whereas “mutation frequency” is the frequency at which a particular mutant is found in the population.

“Hypermutation” or “increased mutation rate” or “increased mutation frequency” refers to the mutation of a nucleic acid in a cell at a rate above background. Preferably, hypermutation refers to a rate of mutation of between 10⁻⁵ to 10⁻³/base/generation. This is greatly in excess of background mutation rates, which are of the order of 10⁻⁹ to 10⁻¹⁰/base/generation (DRAKE et al. 1998).

The term “constitutive hypermutation” refers to the ability of certain cell lines to cause alteration of the nucleic acid sequence of one or more specific sections of endogenous or transgene DNA in a constitutive manner, that is without the requirement for external stimulation. Generally, such hypermutation is directed. In cells capable of directed constitutive hypermutation, sequences outside of the specific sections of endogenous or transgene DNA are not subjected to mutation rates above background mutation rates. The sequences which undergo constitutive hypermutation are under the influence of hypermutation-recruiting elements, as described further below, which direct the hypermutation to the locus in question. Thus in the context of the present invention, target nucleic acid sequences, into which it is desirable to introduce mutations, can be constructed, for example by replacing V gene transcription units in loci which contain hypermutation-recruiting elements with another desired transcription unit, or by constructing artificial genes comprising hypermutation-recruiting elements.

A wide variety of proteins have been subject to random mutation procedures to generate proteins that selectively bind substances. Those of skill will recognize proteins with a reasonable potential for generating such binding. As with many antibodies, scaffold proteins can be composed of subunit proteins. These are “scaffold proteins.” Scaffold proteins that have been used in the past include without limitation immunoglobulin heavy chain or light chain variable regions, combinations of light and heavy chains including Fab fragments, Anticalins, fibronectin type III domain (e.g., Adnectins), Designed Ankyrin Repeat Protein (DARPins), Centyrins, and the like.

A “scaffold protein library” is a library of genetically diverse scaffold proteins. For example, the library can encode Adnectins.

The cytidine deaminase used in the yeast cell embodiments is “effective to contribute to a mutagenic process for inducing a library of binding scaffold proteins from the yeast cell.” This means that, where used alone or in combination with other mutation-inducing circumstances, the cytidine deaminase contributes a statistically meaningful increase in creating binding scaffold protein mutant DNAs.

A surface, such as for example in a polystyrene multititer plate, has a substance “bound” thereto if its association with the surface is strong enough to allow cell panning. The binding can be, but is not necessarily, covalent.

A “color marker” has optical density (in a frequency band) or fluorescence directly, has enzymatic activity that generates the same, or is adapted to selectively bind one or more substances (e.g., biotin) such that eventually in the binding tree substances directly have or enzymatically generate optical density or fluorescence.

The meaning for “identity” for polypeptides is as follows: Polypeptide embodiments (including as components of methods or yeast cell systems) further include an isolated polypeptide comprising a polypeptide having at least about 50, 60, 70, 80, 85, 90, 95, 97 or 100% identity to a polypeptide “Reference Sequence” (e.g. SEQ ID NOs:1, 2, 3, 4 or 5), wherein said polypeptide sequence may be identical to the Reference Sequence or may include up to a certain integer number of amino acid alterations as compared to the Reference Sequence, wherein said alterations are selected from the group consisting of at least one amino acid deletion, substitution, including conservative and non-conservative substitution, or insertion, and wherein said alterations may occur at the amino- or carboxy-terminal positions of the Reference Sequence or anywhere between those terminal positions, interspersed either individually among the amino acids in the Reference Sequence or in one or more contiguous groups within the Reference Sequence, and wherein said number of amino acid alterations is determined by multiplying the total number of amino acids in the Reference Sequence by the integer defining the percent identity divided by 100 and then subtracting that product from said total number of amino acids in the Reference Sequence, or:

nₐ<xₐ−(xₐ·y),

wherein nₐ is the number of amino acid alterations, xₐ is the total number of amino acids in the Reference Sequence, y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and is the symbol for the multiplication operator, and wherein any non-integer product of xₐ and y is rounded down to the nearest integer prior to subtracting it from xₐ.

By way of example, a polypeptide sequence of the present invention may include a contiguous segment of sequence that is identical to the Reference Sequence, that is it may be 100% identical, or it may include up to a certain integer number of amino acid alterations as compared to the Reference Sequence such that the percent identity is less than 100% identity.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more. If there are two ranges mentioned, such as about 1 to 10 and about 2 to 5, those of skill will recognize that the implied ranges of 1 to 5 and 2 to 10 are within the invention.

### II. Methods and Compositions for Diversifying Polypeptide Libraries in Yeast

Cost effective and accelerated methods for antibody discovery will have broad impact on developing diagnostic, research and therapeutic antibodies. Currently ex-vivo non-mammalian approaches for generating antibodies such as phage display (HAWKINS et al. 1992), yeast surface display (BODER and WITTRUP 1997; BODER and WITTRUP 2000), ribosome display (HANES and PLUCKTHUN 1997; HE and TAUSSIG 1997) RNA display (REIERSEN et al. 2005), and mammalian cell display (BEERLI et al. 2008) are not intrinsically capable of affinity maturation because they lack the capacity to effect somatic hypermutation.

Methods that can be potentially useful for antibody discovery are set forth in Table 1.

TABLE 1
Comparison of Abzyme's antibody discovery
platform with other technologies

| | | | In vitro | Antigen | Ease of |
|---|---|---|---|---|---|
| Technology | | | Matura- | limita- | applica- |
| (references) | Speed | Cost | tion | tion | tion |
| SDALib | High | Low | Yes | No | Yes |
| Animal approach | Low | High | Yes | Yes | No |
| Phage display | Moderate | Low | No | No | No |
| Yeast Display | Moderate | Low | No | No | No |
| Ribosomal Display | Moderate | Low | No | No | No |
| Mammalian cell | Moderate | Moderate | No | No | No |
| display | | | | | |
| Mammalian cell | Moderate | Moderate | Yes | No | Yes |
| display + AID | | | | | |

Numerous techniques to generate antibodies were evaluated. As can be seen from Table 1 only SDALib technology described herein meets the desired criteria of cost, speed, self-maturation, no antigen limitation and ease of application. While a number of in vitro techniques can generate antibody, for maturation they require additional steps including in vitro error-prone PCR and library sub-cloning. Antibody maturation by error-prone PCR followed by sub-cloning is easily doable if the antibody is expressed by a single gene such as in the single domain (human VH or camelid VHH) or in the single-chain variable fragment (scFv) formats. When antibodies consist of separate light and heavy chain genes, error-prone PCR sub-libraries have to be constructed for each antigen-specific clone to maintain heavy-light chain pairing. Otherwise random pairing of a light chain from one active antibody with a heavy chain from a different clone will not likely generate again a target-specific antibody.

There is a continuing need in the art for better developing antibodies. This invention is a novel technology platform, called Self-Diversifying Antibody Library or SDALib, that comprises a binding moiety (such as a monoclonal antibody) generating system providing a diverse array of binding moieties (such as complete antibodies) in vitro, without using immunization. The invented system can be used for polypeptide library diversification, protein maturation and screening of binder proteins with modified affinity to another molecule. Advantages of this invention include low cost, rapid growth eukaryotic protein expression and a surface display system with ease of culture, culture maintenance, facile manipulation and genetic engineering. Moreover, yeast mating allows combination of antibody heavy chain and light chain libraries to form a single library with highly diverse random H/L combinations. The expression of sea lamprey CDA—the most powerful deaminase mutator in yeast-directed to a DNA target in combination with the chemical supermutagen HAP allows rapid library diversification, and finally the use of diploid and/or polyploid yeast strains protect yeast cells from detrimental genetic damage of the induced mutagenesis due to the presence of two or more copies of essential genes. In combination with Fluorescence Assisted Cell Sorting (FACS) yeast cells expressing functional binders can be quickly identified.

In accordance with the present invention, a yeast-based genetic system and methods of use, thereof are provided to facilitate antibody discovery. The methods provided herein enable the rapid and efficient maturating and isolating antibody clones to an antigen target of interest starting from a naïve antibody library or to improve the activity of a known protein, including antibodies.

### III. Preparation of Nucleic Acid Molecules Encoding the Proteins of the Invention and Uses Thereof in Assay Methods and Kits

### A. Nucleic Acid Molecules

Nucleic acid molecules encoding the expression vectors of the invention can be prepared by two general methods: (1) They can be synthesized from appropriate chemical starting materials, or (2) they can be isolated from biological sources. Both methods utilize protocols well known in the art.

The availability of nucleotide sequence information, for the sea lamprey CDA1, as well as for secretory signals from alpha-mating factor or yeast SUC2 gene facilitates synthesis of DNA constructs containing such sequences. Synthetic oligonucleotides can be prepared by the phosphoramadite method employed in the Applied Biosystems 38A DNA Synthesizer or similar devices. The resultant construct can be purified according to methods known in the art, such as high performance liquid chromatography (HPLC). Long, double-stranded polynucleotides, such as a DNA molecule encoding a construct of the present invention, must be synthesized in stages due to the size limitations inherent in current oligonucleotide synthetic methods. Thus, for example, a 3 kilobase double-stranded molecule can be synthesized as several smaller segments of appropriate complementarity. Complementary segments thus produced can be ligated such that each segment possesses appropriate cohesive termini for attachment of an adjacent segment. Adjacent segments can be ligated by annealing cohesive termini in the presence of DNA ligase to construct the entire 3 kilobase double-stranded molecule. A synthetic DNA molecule so constructed can then be cloned and amplified in an appropriate vector. In alternative embodiments of the invention, the sea lamprey CDA1, yeast AGA2, yeast SUC2 secretory signal and camelid VHH genes can be substituted with similar genes with functional homology from other biological sources. In the PmCDA1 example, suitable candidate genes for such substitution include, without limitation, lamprey cytidine deaminase mutated (modified or altered cytidine deaminases), derivatives such as a CDA1 hybrid with ER DNA binding domain (ER-DBD), which also has high mutator phentotype once expressed in yeast defective in Uracil-DNA glycosylase. In addition, one can replace PmCDA1 with cytidine deaminase from other species, including but not limited to human AID.

Yeast SUC2 secretory signal of the invention used for promoting protein secretion can be derived from different species not limited to S. pombe and K. lactis. It can be substituted with yeast alpha mating factor secretory signal that also functions as secretory signal.

Similarly, the DNA binding domain of human estrogen alpha receptor (ER) of the invention can be derived from different species. The ER DNA binding domain has been used in the studies described herein. However, ER DNA binding domain (ER-DBD) from rat or mouse can have altered properties that can make them more robust and efficient than the human ER-DBD. The purpose of using ER-DBD fused with CDA is to recruit CDA to the ERE response sequence that is operably linked to VHH encoding genes. Thus, in this situation, the functional activity of ER-DBD can be replaced with other DNA binding domains to recruit CDA to a respective response element.

The interaction between a DNA binding domain and a DNA binding protein recognition sequence can be used to direct lamprey cytidine deaminase to a specific nucleic acid sequence. One way of directing mutation in this way is described as follows: an expression construct for expressing a fusion protein comprising lamprey cytidine deaminase with the estrogen receptor DNA binding domain (ERD) is created. The expression construct is expressed in yeast. The yeast host cell is also engineered such that the desired target gene is also operably linked to a short ERD recognition sequence. Those of skill will recognize that other DNA-binding moieties can be used to provide localization. A useful binding moiety is an ERDBD with about 85% or more sequence identity with the human ERDBD of SEQ ID NO. 14, or with about 90 or 95% or more sequence identity with one of SEQ ID NOs. 10, 11, 12 or 14.

Nucleic acid sequences encoding the components of the expression plasmids of the invention can be isolated from appropriate biological sources using methods known in the art. For example, RNA isolated from a mammalian or insect cell can be used as a suitable starting material for the generation of cDNA molecules encoding the different receptor proteins.

In accordance with the present invention, nucleic acids having the appropriate level of sequence homology with the protein coding region of the DNA molecules of the present invention can be identified by using hybridization and washing conditions of appropriate stringency. For example, hybridizations can be performed at 37° C. to 42° C. for at least six hours. Targets with greater than about 75% sequence identity would be observed using a hybridization temperature of 42° C. Such a sequence would be considered substantially homologous to the sequences of the present invention.

The nucleic acids of the invention can also be used as starting materials for the generation of sequence variants or truncation mutants of the nucleic acids of the invention using any number of synthetic and molecular biologic procedures well known in the art including, but not limited to, truncation at available restriction sites and site-directed mutagenesis techniques. Particular mutations can give rise to receptor proteins with altered characteristics such as increased or decreased ligand binding activity.

### B. Fusion Proteins

In one embodiment of the invention, the antibodies of the invention are expressed in yeast as fusion proteins having yeast secretory signal at the N-terminus. Secretory signal fusion at the N-terminus of the protein facilitates extracellular secretion of the protein following translation. After translation of recombinant proteins, the secretory signals at the N-terminus are cleaved by the host secretory pathway proteases and native proteins are released. It is widely known that the presence of the secretory signals results in secretion of proteins of interest in yeast (KJAERULFF and JENSEN 2005).

In embodiments of the invention, the antibodies of the invention are expressed in yeast as fusion proteins with yeast membrane anchor protein AGA2 at their C-termini. The protein membrane anchor functions to retain secreted antibodies or polypeptides on the yeast cell surface (BODER and WITTRUP 1997) therefore accessible to an antigen of interest located extracellularly.

In embodiments of the invention, the antibodies of the invention are expressed in yeast as ER-DBD fusion proteins. ER-DBD fusion can enhance but is not necessary for hypermutation in antibody-encoding DNA regions coupled with a ER-DBD response element (ERE).

### C. Assay Methods and Kits

In yet another embodiment of the invention, assays are provided wherein intact cells expressing a protein of interest are grown in cell culture media containing nucleotide base analogue molecules and molecules to induce expression of cytidine deaminase. After a suitable time period, the diversification of a gene or protein of interest is measured. Such diversification of a gene or protein of interest can be quantitated in any number of ways. For example, such cell systems can utilize a reporter system in which the production of the reporter signal is dependent on enzymatic or fluorescence or binding activity of the protein of interest. Numerous reporters can serve equally well in this application including but not limited to, beta-galactosidase, alkaline phosphatase, fluorescent green protein, antibody, protein scaffold and the like. Inactivation or activation of the gene of interest can be also measured as forward and reverse mutation rates. For example, mutation in the arginine permease encoding gene CAN1 confers resistance to canavanine (LANG and MURRAY 2008). The gene diversification level can be measured by Next Generation Sequencing. Furthermore, the methods of the invention can be practiced in bacterial, fungal, insect, avian, mammalian or plant cells. However, yeast-based cell systems are preferred due to low cost and the feasibility of growing yeast cells in plastic devices.

Assays for screening binders to a target of interest are also provided. Diversification of binders in the cell-based system can be followed by isolation of cells expressing modified binders reactive to a target by means of biological panning or fluorescence-activation cell sorting (FACS). Isolation of binders reactive to a target of interest can be performed as described previously (CHAO et al. 2006) In another aspect, the invention includes kits to facilitate the use of the compositions and methods disclosed herein. Exemplary kits include the expression plasmids, yeast-based scaffold libraries and yeast strains of the invention, and/or variants thereof. Also provided are cell culture media, compounds and protocols for use of the compositions of the invention for the particular application and the necessary reagents to carry out the application are also provided. Such reagents can include, but not be limited to, buffers, solvents, media and solutions.

The following protocols are provided to facilitate construction of the expression plasmids for use in the methods and kits of the present invention.

### Yeast Media, Strains, Plasmids, Antibody Library

Standard yeast and E. coli media were prepared as described in detail (CHAO et al. 2006). For example, YPD composed of Yeast Extract (20 grams per liter), Peptone (20 g/liter), Dextrose (20 g/Liter) is preferred for use in the invention as most yeast strains grow in this media. Yeast selective media (complete-drop out) used to maintain plasmids is composed of yeast nitrogen base 1.7 g/L, ammonium sulfate 5 g/L, dextrose 20 g/L, different amino acids and other supplements depending on the requirements of the particular yeast strain. For example if the yeast plasmid contains the LEU2 marker, the leucine is dropped out from the media in order to select the plasmid. Other buffered selective media such as SDCAA glucose media and SGRCAA galactose media were prepared as described in detail (CHAO et al. 2006).

Yeast strains suitable for use in the present invention include the yeast strains of opposite mating types A101: MatA ura3-52 trp1Δ63 leu2 pep4-3 prb1-22 prc1-407 ung1::HygB ham1::KanMX; A169: Mat Alpha Gal1p-AGA1::URA3 ura3-52 trp1::NatMX leu2-Δ200 his3-Δ200 lys2Δ pep4::Zeo^{R} prbΔ1.6R can1 ung1::HygB ham1::KanMX and A170: MatA Gal1p-AGA1::URA3 ura3-52 trp1-Δ63 Gal1p-PmCDA1::LEU2 leu2 his3::zeo^{R} pep4-3 prb1-22 prc1-407 ung1::HygB ham1::KanMX.

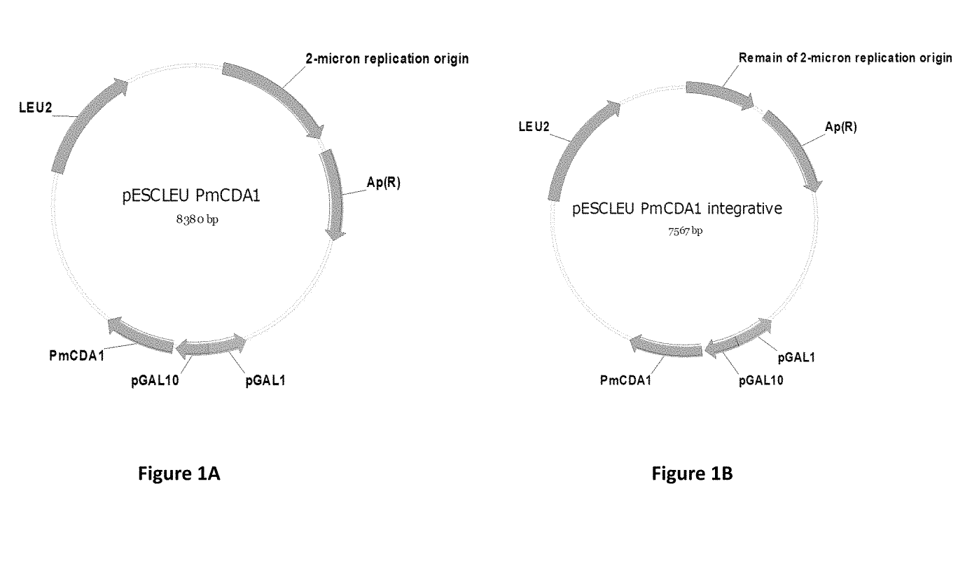

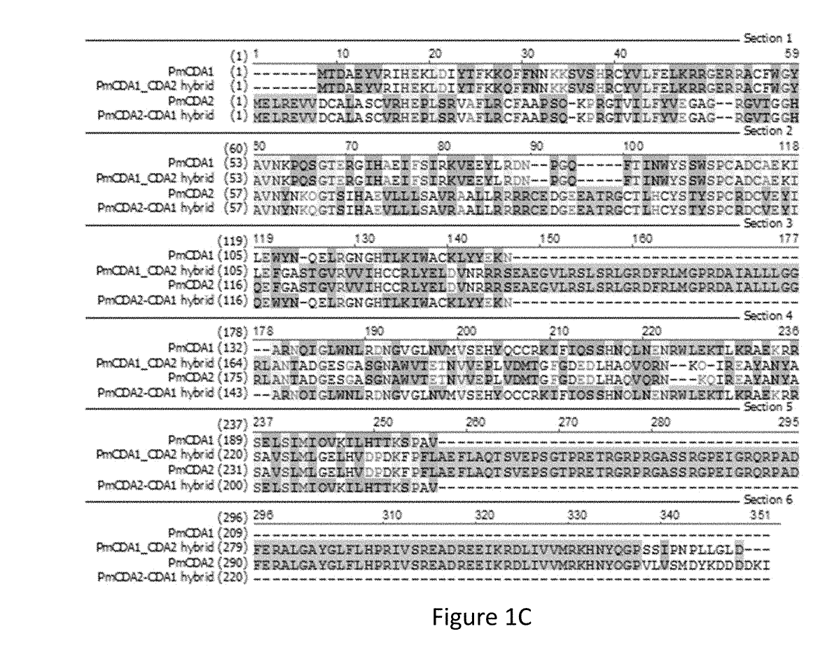

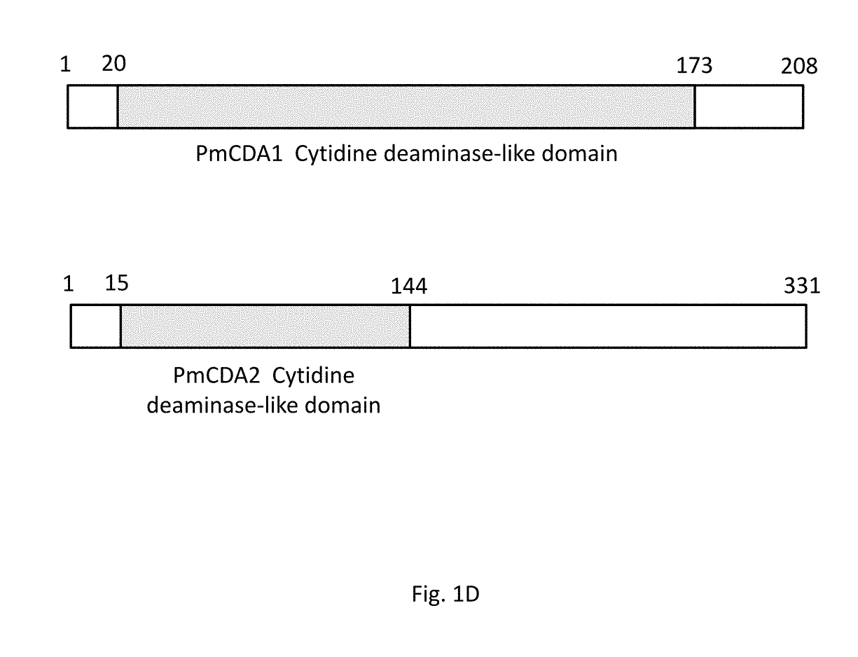

A series of yeast expression plasmids pESCLEU-PmCDA1, pESCLEU-PmCDA2, pESCLEU-PmCDA2/1 and pESCLEU-hAID have been constructed for expressing sea lamprey cytidine deaminases and their derivatives as well as human AID based on the pESCLEU plasmid backbone (Agilent Technologies). FIG. 1A presents a representative plasmid pESCLEU-PmCDA1 (with gal-induced expression of CDA1). All plasmids are yeast-E. coli multicopy shuttle plasmids containing LEU2 as a yeast transformation marker. Respective integrative vectors have been obtained from these plasmids by removing the SnaBI-BsmBI region of the yeast 2-micron replicative origin. FIG. 1B presents a representative plasmid pESCLEU-PmCDA1 integrative. A plasmid expressing cytidine deaminase is introduced into A101 by yeast transformation selecting for Leu⁺ transformants. CDA-expressing transformants are mated with A169-derived strains that contain constructs expressing a gene of interest to form diploids. Diversification of the gene of interest will be accomplished by culturing in media containing galactose as a sole carbon source or media containing base analogues.

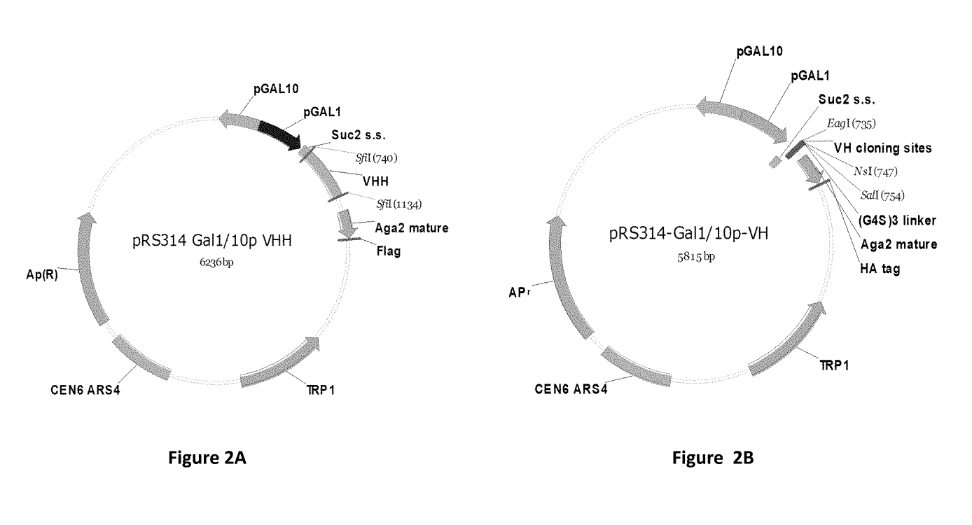

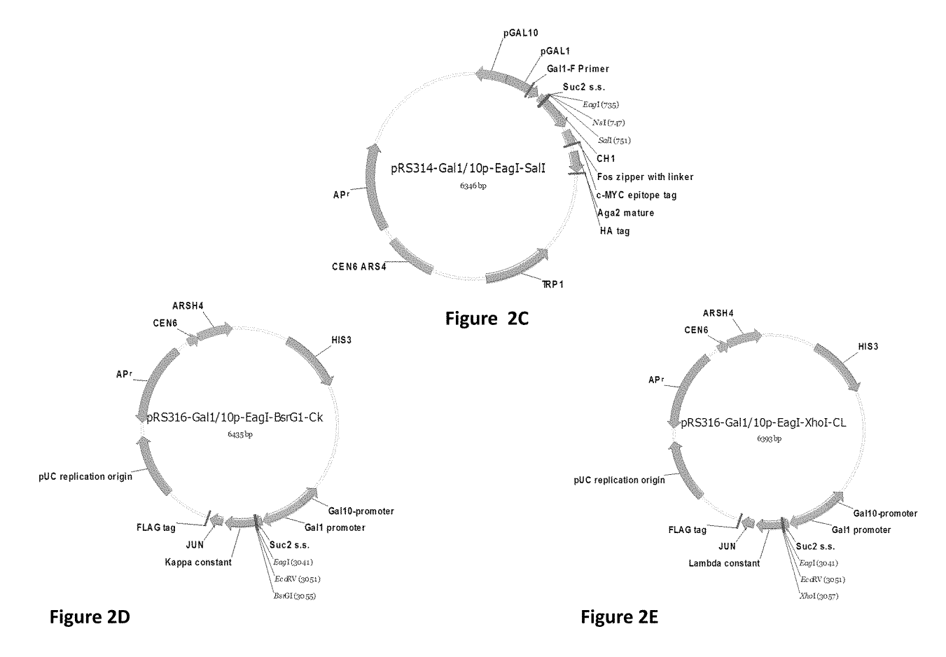

A series of yeast expression plasmids for expression and display of a protein of interest on the yeast cell surface were constructed based on the yeast-E. coli single copy plasmid pRS314 (SIKORSKI and HIETER 1989). All plasmids are yeast-E. coli centromeric shuttle plasmids containing TRP1 as a yeast transformation marker. FIG. 2 depicts an exemplary plasmid pRS314_Gal1/10p_VHH of the invention that is suitable for expression and display of camelid heavy chain variable domains on the yeast cell surface. pRS314_Gal1/10p_VHH is comprised of the centromeric plasmid pRS314 (SIKORSKI and HIETER 1989), S. cerevisiae bi-directional galactose inducible promoter Gal1/10, yeast Suc2 (invertase) secretory signal, SfiI-SfiI cloning sites, linker, membrane anchor AGA2 and FLAG tag at its C-terminus. pRS314-Gal1/10p-VH (FIG. 2B) used for expressing human VH has a similar structure containing the Gal1/10 promoter, yeast Suc2 (invertase) secretory signal, EagI-SalI cloning sites, linker membrane anchor AGA2 and HA tag at its C-terminus. Proteins of interest such as antibody heavy chain or light chain variable regions, protein scaffolds including, but not limited to Anticalins, fibronectin type III domain—Adnectins, Designed Ankyrin Repeat Protein or DARPins and Centyrins can be expressed and displayed on the yeast cell surface using the two vectors mentioned above.

When a protein of interest is a heterodimer such as an antibody Fab fragment, it requires two plasmids to express both partners simultaneously. For example, to express and display antibody Fab fragments, two yeast-E. coli centromeric plasmids have to be constructed to express both heavy chain and light chains. For expression of heavy chain, the yeast-E. coli centromeric plasmid pRS314-Gal1/10p-EagI-SalI plasmid (FIG. 2C) that contains the yeast Gal1/10 promoter, Suc2 invertase secretory signal, cloning sites EagI and SalI for heavy chain VH cloning, CH1 constant domain, leucine zipper FOS followed with HA tag was constructed. The plasmid contains a TRP1 yeast transformation marker. For expression of light chain, a series of plasmids derived from plasmid pRS316 (SIKORSKI and HIETER 1989) that contains a HIS3 transformation selection marker were constructed. pRS316-Gal1/10p-EagI-BsrG1-Ck for kappa light chain (FIG. 2D) and pRS316-Gal1/10p-EagI-XhoI-CL for lambda light chain (FIG. 2D) were constructed. These two plasmids each contains a yeast Gal1/10 promoter, Suc2 invertase secretory signal, cloning sites EagI/BsrGI or EagI/XhoI for light chain variable cloning, Kappa or Lambda light chain constant region, respectively, leucine zipper from Jun to facilitate dimerization followed with FLAG tag for display and expression monitoring. Heteromeric formation is possible via formation of heavy chain and light chain constant domains facilitated by the leucine zipper Fos/Jun. The pRS316-based plasmid contains a HIS3 yeast transformation marker.

To display and diversify Fab fragments, heavy chain and light chain variable domains are cloned in frame at the cloning sites in the respective vectors described above. Heavy chain vectors are introduced into the A169 yeast strain by yeast transformation selecting colonies growing in media without tryptophan. Light chain vectors are introduced into the A170 strain that already contains the integrated PmCDA1 vector (FIG. 1B) by transformation selecting colonies growing in media without histidine. A diploid is obtained by mating Trp⁺ and His⁺ transformants and selecting Trp⁺ His⁺ colonies. Genes expressing heavy chain and light chain variable domains are diversified by growing diploids in media containing galactose as a sole carbon source to induce both antibody encoding genes and CDA1 encoding gene.

Also provided is a camelid VHH library constructed using the yeast-E. coli expression vector pRS314-Gal1/10-VHH (FIG. 2A). Complementary DNA (cDNA) derived from camelid leukocyte mRNA encoding heavy chain variable domains have been cloned in frame with the secretory signal and AGA2. cDNA library construction is well known in the art. The camelid VHH library has been introduced into the A169 yeast strains using Trp⁺ selection. The final self-diversifying camelid antibody library was obtained by mating A169 library with the A170 yeast strain containing the CDA gene integrated.

### Diversification Methods

A diploid host cell containing a first DNA construct having a nucleic acid molecule encoding a protein that is subjected for diversification and a second DNA construct having a nucleic acid molecule encoding cytidine deaminase will undergo diversification by two means either performed separately or in combination.

In the first means of diversification a host cell containing constructs of the invention is continuously grown in yeast selective media that contains promoter inducers including but not limited to (1) galactose (20 g/L) as a sole carbon source to induce the Gal1/10 promoter or (2) copper at concentrations of 100 micro molar to 1 mM to induce the Cup1 promoter. Under such conditions cytidine deaminase is expressed. Produced cytidine deaminases will convert C to U via deamination in transcriptionally active genes including a gene of interest.

In the second means of diversification, a host cell containing constructs of the invention is continuously grown in yeast selective media containing base analogues including, but not limited to 6N-hydroxylamine purine or HAP. During replication the base analog 6-N-hydroxylaminopurine (HAP) induces bidirectional GC->AT and AT->GC transitions (SHCHERBAKOVA and PAVLOV 1993).

As gene diversification occurs via hypermutation that is active during cell division via DNA replication, the level of diversification obtained is directly related to numbers of mutations in a gene of interest that accumulate as cells grow. Therefore the longer cells undergo diversification, the more mutations will accumulate.

### EXAMPLES

Specific embodiments according to the methods of the present invention will now be described in the following examples. The examples are illustrative only, and are not intended to limit the remainder of the disclosure in any way.

Where a sentence states that its subject is found in embodiments, or in certain embodiments, or in the like, it is applicable to any embodiment in which the subject matter can be logically applied.

### Example 1

Isolation of VHH Antibody Clones from a Naïve VHH Antibody Library

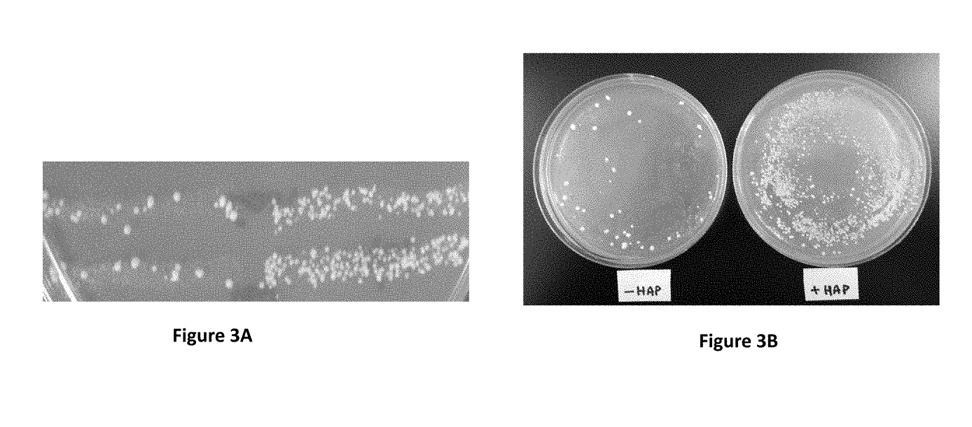

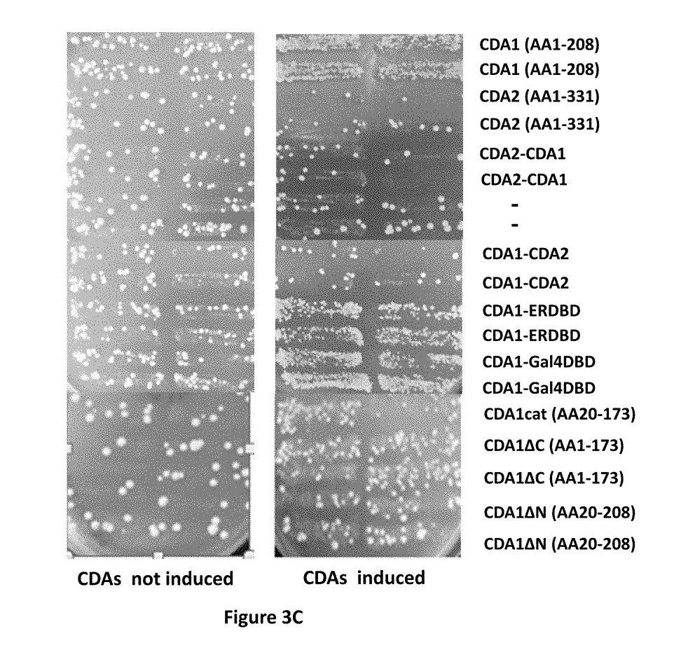

In accordance with the present invention, compositions and methods are provided for diversification of a polypeptide library and isolation of binders from a diversified polypeptide library to a target of interest. It has previously been demonstrated that the overexpression of sea lamprey CDA1 can cause mutations in yeast strains defective in Uracil DNA-glycosylase (UNG1 mutant) (MAYOROV et al. 2005b) and FIG. 3A. In addition, 6-N-hydroxylaminopurine (HAP) causes substitution mutations in yeast HAM1 mutant strains (NOSKOV et al. 1996) and FIG. 3B. Thus, overexpression of sea lamprey CDA1 in yeast ung1 mutants or the presence of the HAP mutagen in yeast HAM1 mutants either alone or in combination can serve as mutation causing factors for diversifying genes encoding polypeptides of significance.

Using prior art, mRNA from camelid blood leukocytes have been isolated, converted into cDNA and DNA regions encoding camelid immunoglobulin heavy chain variable domains have been cloned at the SfiI-SfiI sites of the vector in frame with secretory signal and the membrane anchor AGA2 as presented in the FIG. 2A. Thus, a naïve VHH library has been created using the yeast-E. coli expression vector that is capable of replication in both E. coli and yeast. The VHH naïve library represents a first construct mentioned in numbered Embodiment A1. The yeast surface display VHH library has been introduced into the A169 yeast strain by electroporation and selection for Trp⁺ cells. More than 5×10⁷ of Trp⁺ transformants were obtained. 10¹⁰ cells of the haploid library were mated with 10¹⁰ cells of the A170 strain that contains the integrated AID gene. The diploids were selected in yeast minimal media supplemented with histidine. The mating efficiency was 20% according to plating efficiency. It is estimated that 2×10⁹ diploids were formed.

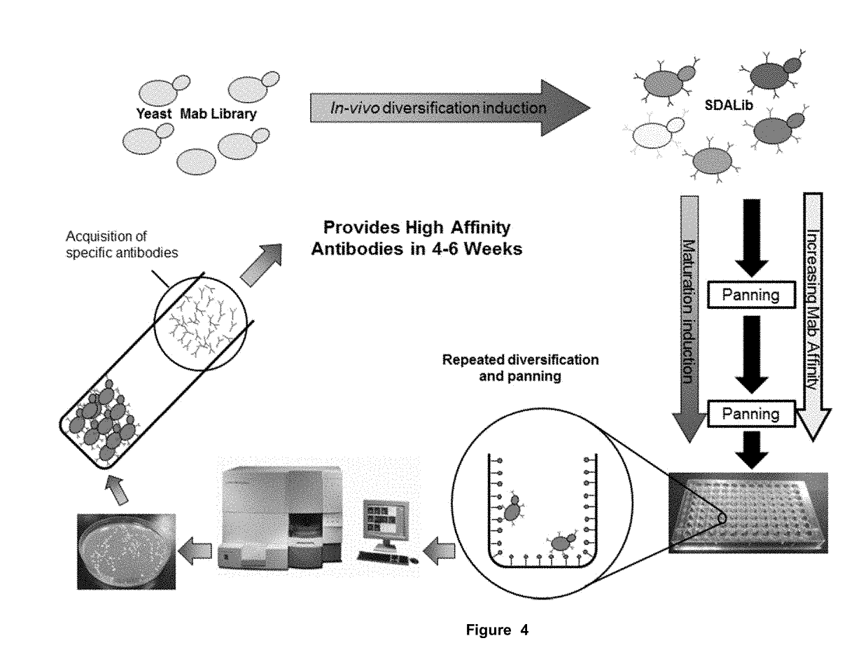

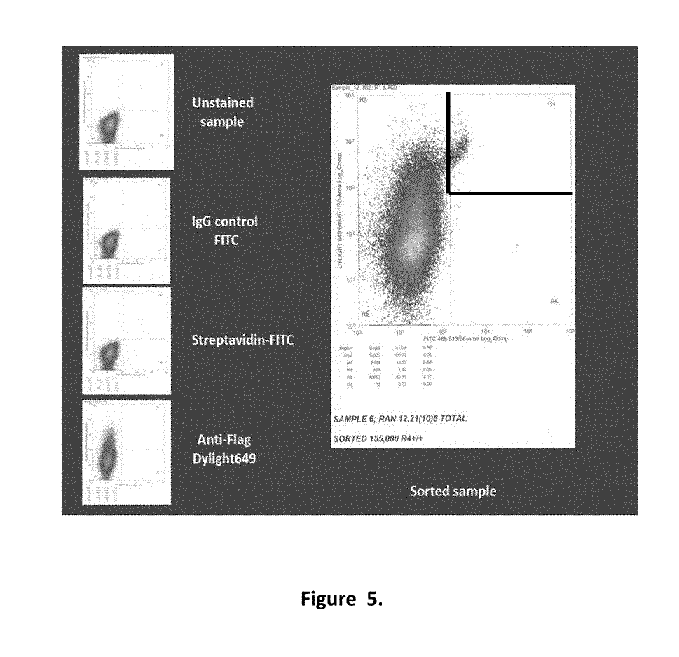

The diploid VHH library underwent repeated diversification induction by continuous propagation at 20° C. for 24 hours in yeast buffered-media SGRCAA selective media (CHAO et al. 2006) supplemented with galactose in the presence of HAP at a concentration 25 μg/mL followed by target-specific panning. FIG. 4 presents the scheme used for VH library diversification and antigen-directed enrichment via panning. In this example, influenza H5N1 neuraminidase was used as a target. The naïve camelid VHH library in yeast diploids underwent self-diversification. Yeast clones expressing VHH to H5N1 neuraminidase were enriched using the biotinylated H5N1 neuraminidase (H5N1 A/Thailand/1(KAN-1)/2004) purchased from Sinobiologicals and streptavidin-magnetic particles or anti-biotin beads immobilized on Miltenyi LD columns/QuadraMacs (Miltenyi) according to the system manual. Alternating magnetic beads (streptavidin-magnetic bead and anti-biotin beads) were used to eliminate clones reactive to the secondary reagents. After four cycles of diversification and panning, yeast cells expressing VHH to N1-NA were sorted twice by Fluorescence-Activated Cell Sorting (FACS). FIG. 5 shows one FACS cell sorting to isolate yeast clones expressing VHH antibodies to neuraminidase.

### Example 2

DNA Sequence Analysis of Isolated VHH Antibody Clones

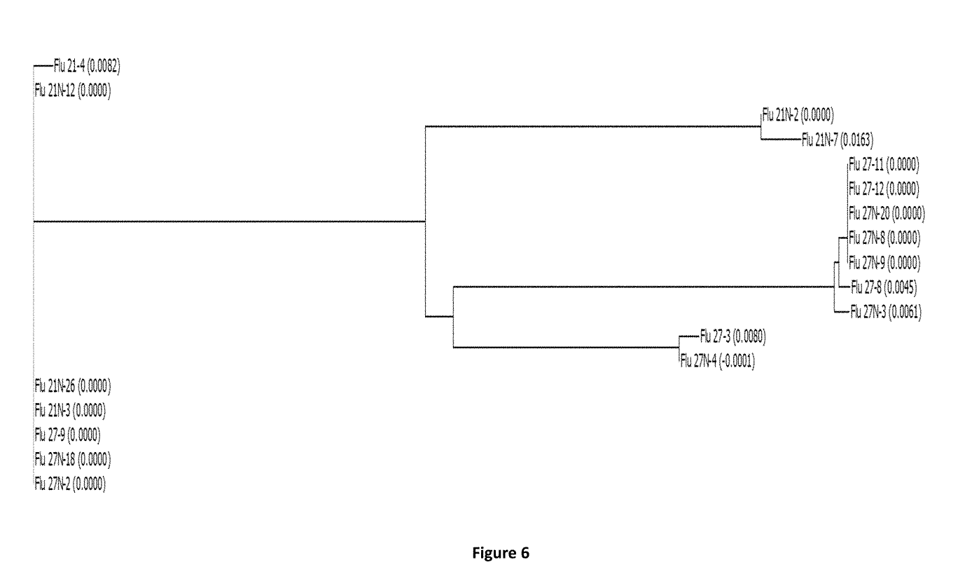

Total plasmid DNA from a pool of FACS-sorted yeast cells was isolated and VHH encoding genes were PCR amplified for subsequent recloning into the pET22 (b) expression vector and the resulting individual E. coli clones were analyzed for anti-influenza H5N1 neuraminidase (“N1 NA”) activity. Protein expression was induced using autoinduction media as described (STUDIER 2005) and cell shockate was subjected to direct ELISA for binding to N1-NA. VHH from candidate positive clones were purified using Ni-NTA resins and the activity with N1-NA was confirmed by direct ELISA. Eighteen VHH antibodies that are reactive to H5N1 NA were isolated and sequenced. Sequence alignment (FIG. 6) showed not only the diversity of these clones, but also that several clones likely originated from one parent VHH via mutation. For example, clones 27-11 and 27-8 differ by only in 1 amino acid in CDR3.

### Example 3

Purification and Characterization of Isolated VHH Antibodies

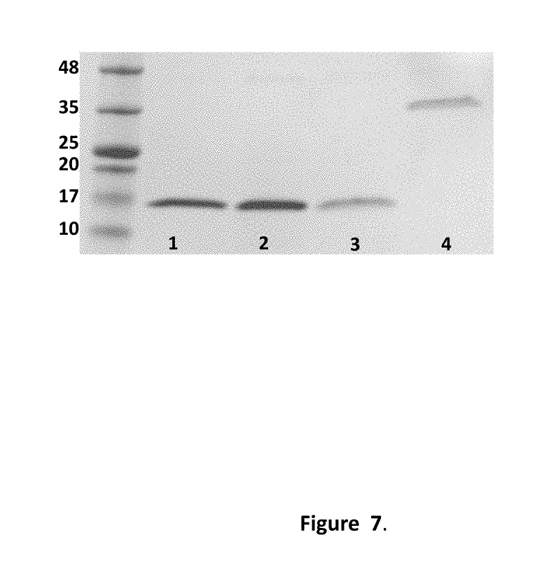

The periplasmic expression and protein purification of recombinant FLAG-His6-tagged VHH was performed as described (CONRATH et al. 2001). The purity of the proteins was confirmed by SDS-PAGE (FIG. 7). The protein concentration was determined spectrophotometrically at 280 nm using the computed extinction coefficient of each VHH.

A DNA construct was cloned to generate the bivalent proteins VHH1-LH-VHH2 tailed by Flag-tag and 6×HIS tag. “LH” stands for “long hinge” and is the structural upper hinge of the llama IgG2, AHHSEDPSSKAPKAPMA SEQ ID NO. 15 (Vu et al. 1997). The N-terminal antibody fragment was cloned in frame with the pelB signal sequence for potential periplasmic localization. VHH proteins were expressed and purified using Ni-NTA resins as described previously (CONRATH et al. 2001). FIG. 7 presents the purified monovalent (VHHm) and bivalent (VHHb) antibodies.

Table 2 presents the ELISA data of purified monovalent and bivalent antibodies. Antibody titers to NA were measured by ELISA using microplates coated with (0.1 ug/well) NA antigens and serial dilutions of antibody. Bound VHH antibodies were detected with HRP conjugated mouse anti-FLAG IgG (Rockland Immunochemicals). NA inhibition activity of monospecific and multivalent antibodies was measured using a Neuraminidase assay (BioAssay Systems).

TABLE 2
Representative ELISA data showing binding activity of
VHH antibodies isolated in the present invention to
the H5N1 influenza neuraminidase in ELISA assays.

Targets

| Nanobody | Dilution | H5N1 | H1N1 | H3N2 |
|---|---|---|---|---|
| 27-8 | 1:100 | 2.48 | 1.89 | 1.08 |
| h27-8 | 1:10 | 3.27 | 1.93 | 1.59 |
| h27-8 | 1:100 | 2.53 | 1.56 | 1.28 |
| h27-8GL | 1:10 | 2.27 | 1.62 | 1.34 |
| h27-8GL | 1:100 | 1.78 | 1.21 | 1.09 |
| h27-8GL/h27-8GL | 1:10 | 3.73 | 2.44 | 1.67 |
| h27-8GL/h27-8GL | 1:100 | 2.15 | 1.31 | 1.13 |

Note: Shown is ELISA data of the original VHH 27-8 and three different humanized VHH h27-8, h27-8GL and h27-8GL/h27-8GL against three different neuraminidase targets. The first three VHH are monovalent, the last one is a bivalent VHH. Shown is the reactivity compared to control protein used as target (BSA).

### Example 4

### Mutation Phenotypes of Sea Lamprey CDA Variants and Derivatives

In accordance with the present invention, a polypeptide library is diversified by overexpression of sea lamprey CDA1 and its variants. FIG. 3C. demonstrated that the overexpression of sea lamprey CDA1 and some of its variants can cause mutations in yeast strains defective in Uracil DNA-glycosylase (UNG1 mutant). It is noted that only overexpression of sea lamprey CDA1, but not sea lamprey CDA2 nor CDA2-CDA1 and CDA1-CDA2 hybrid constructs causes forward mutations in the CAN1 reporter gene. While overexpression of cytidine deaminase-like catalytic domain (AA20-173) of CDA1 shows mutator effect, the mutation rate caused by overexpression of CDA1 cytidine deaminase-like catalytic domain is significantly less than that caused by the overexpression of full length CDA1. Deletion of CDA1 N-terminus (AA 1-19) or C-terminus (AA174-208) significantly reduces its mutator effect. Addition of human estrogen receptor DNA binding domain or yeast Gal4 DNA binding domain to PmCDA1 at its C-terminus does not reduce the PmCDA1 mutator effect on CAN1 reporter (FIG. 3C). Table 3 summarizes the effect on CAN1 forward mutations caused by overexpression of sea lamprey CDA1 and CDA2 and their variants in yeast strain defective in Uracil DNA-glycosylase.

TABLE 3
Mutator effect scores of overexpression sea lamprey CDA1,
CDA2 and their variants in yeast strain defective in Uracil
DNA-glycosylase using CAN1 forward mutation reporter.

| Number | Constructs | Score |
|---|---|---|
| 1 | Empty vector, no sea lamprey CDA | + |
| 2 | PmCDA1 | +++++ |
| 3 | PmCDA2 | + |
| 4 | PmCDA2-CDA1 hybrid | + |
| 5 | PmCDA1-CDA2 hybrid | + |
| 6 | PmCDA1 cytidine deaminase-like catalytic | ++ |
| | domain (AA20-173) | |
| 7 | PmCDA1 with AA1-19 N-terminus deletion | ++ |
| 8 | PmCDA1 with AA174-208 C-terminus deletion | ++ |
| 9 | PmCDA1-ERDBD CDA1 fused with Gal4 DNA | +++++ |
| | binding domain | |
| 10 | PmCDA1-ERDBD CDA1 fused with ER DNA | +++++ |
| | binding domain | |

The invention described herein is of a composition and method for diversifying polypeptide libraries in yeast. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

The invention further relates to the following numbered embodiments:

### Embodiment A1

A system and method for diversifying a protein of interest comprising: a) a first DNA construct having a nucleic acid molecule encoding a protein of interest operably linked to a promoter; b) a second DNA construct having a nucleic acid molecule encoding a second polypeptide operably linked to a promoter; c) a host cell comprising said first and second constructs, diversification of said first construct being dependent upon the expression of the second construct effectuated by said host cell genetic background, and d) diversification of said first construct is also effectuated by said host cell genetic background and the presence of mutation-inducing base analog chemicals.

### Embodiment A2

A system as in one of the “A” Embodiments, wherein said first polypeptide is selected from a group of proteins with measurable functional activity including but not limited to target binding and enzyme activity.

### Embodiment A3

A system as in one of the “A” Embodiments, wherein said first polypeptide is selected from a group of immunoglobulin heavy chain or light chain variable regions or polypeptide scaffolds including, but not limited to Anticalins, fibronectin type III domain—Adnectins, Designed Ankyrin Repeat Protein or DARPins and Centyrins.

### Embodiment A4

A system as in one of the “A” Embodiments, wherein said immunoglobulin heavy chain or light chain variable regions can be from any organism.

### Embodiment A5

A system as in one of the “A” Embodiments, wherein said first DNA construct is operably linked to a motif for a DNA binding protein domain.

### Embodiment A6

A system as in one of the “A” Embodiments, wherein said motif for a DNA binding protein domain is including, but not limited to estrogen response elements.

### Embodiment A7

A system as in one of the “A” Embodiments, wherein said second DNA construct encodes lamprey Petromyzon marinus cytidine deaminase, PmCDA1 protein (Seq. 1, Uniprot # A5H718).

### Embodiment A8

A system as in one of the “A” Embodiments, wherein said DNA-modifying protein is a variant or derivative of sea lamprey Petromyzon marinus cytidine deaminase 1.

### Embodiment A9

A system as in one of the “A” Embodiments, wherein said Petromyzon marinus cytidine deaminase is fused with a DNA binding domain.

### Embodiment A10

A system as in one of the “A” Embodiments, wherein said DNA binding domain includes, but is not limited to the DNA binding domain of estrogen receptor and Gal4 transcriptional factor.

### Embodiment A11

A system as in one of the “A” Embodiments, wherein at least one of said promoters in step a) and b) is an inducible promoter selected from the group consisting of galactose-inducible promoters GAL1 and GAL10, temperature regulatable like heat-shock inducible promoter HSP70, and copper-inducible CUP1 promoters.

Embodiment A12. A system as in one of the “A” Embodiments, wherein said mutation-inducing chemical is selected from the group consisting of purine or pyrimidine base analogs.

### Embodiment A13

A system as in one of the “A” Embodiments, wherein said base analogue is 6-N-hydroxylaminopurine (HAP).

### Embodiment A14

A system as in one of the “A” Embodiments, wherein said host cell is a yeast cell.

### Embodiment A15

A system as in one of the “A” Embodiments, wherein said yeast is diploid or polyploid yeast.

### Embodiment A16

A system as in one of the “A” Embodiments, wherein said host genotype includes inactivation of the Uracil-DNA glycosylase encoding UNG1 gene.

### Embodiment A17

A system as in one of the “A” Embodiments, wherein said host genotype includes inactivation of the HAM1 gene.

### Embodiment A18

A method in one of the “A” Embodiments, wherein said host cell is grown in conditions to activate said inducible promoter.

### Embodiment A19

The method in one of the “A” Embodiments, wherein said host cell is grown in the presence of a base analogue.

### Embodiment A20

The method in one of the “A” Embodiments, wherein said a base analogue includes, but is not limited to 6-N-Hydroxylaminopurine.

### Embodiment B1

A yeast cell comprising: (A) a recombinant DNA that constitutively or inducibly expresses a cytidine deaminase comprising sequence with about 90% sequence identity or more with a cytidine deaminase domain of (i) SEQ ID NO. 2 or SEQ ID NO. 4, or (ii) a chimera between the two starting with SEQ ID NO. 3 or SEQ ID NO. 4 sequence and having one transition to end in SEQ ID NO. 1 or SEQ ID NO. 2 sequence, or (iii) a chimera between the two starting with SEQ ID NO. 1 or SEQ ID NO. 2 sequence and having one transition to end in SEQ ID NO. 3 or SEQ ID NO. 4 sequence; and (B) a second recombinant DNA that constitutively or inducibly expresses a binding scaffold protein for presentation on the outer surface of the yeast, wherein the cytidine deaminase as expressed by the first recombinant DNA is effective to contribute to a mutagenic process for inducing mutations in the binding scaffold protein of the yeast cell. Per the “comprising” language, the beginning and end language does not imply that the recombinant DNA, or the expressed protein begins or ends at a particular location.

Embodiment B1a. A yeast cell comprising: (A) a recombinant DNA that constitutively or inducibly expresses a cytidine deaminase comprising sequence with about 90% sequence identity or more with SEQ ID NO. 2, SEQ ID NO. 4, or a chimera between the two starting with SEQ ID NO. 3 or SEQ ID NO. 4 sequence and having one transition to end in SEQ ID NO. 1 or SEQ ID NO. 2 sequence; and (B) a second recombinant DNA that constitutively or inducibly expresses a binding scaffold protein for presentation on the outer surface of the yeast, wherein the cytidine deaminase as expressed by the first recombinant DNA is effective to contribute to a mutagenic process for inducing mutations in the binding scaffold protein of the yeast cell. Per the “comprising” language.

### Embodiment B2

A method of generating a binding activity comprising: (A) cultivating a culture of yeast cells that comprise: (a) a first recombinant DNA that constitutively or inducibly expresses a cytidine deaminase with about 90% sequence identity with a cytidine deaminase domain of (i) SEQ ID NO. 2 or SEQ ID NO. 4, or (ii) a chimera between the two starting with SEQ ID NO. 3 or SEQ ID NO. 4 sequence and having one transition to end in SEQ ID NO. 1 or SEQ ID NO. 2 sequence, or (iii) a chimera between the two starting with SEQ ID NO. 1 or SEQ ID NO. 2 sequence and having one transition to end in SEQ ID NO. 3 or SEQ ID NO. 4 sequence; and (b) a second recombinant DNA that constitutively or inducibly expresses a binding scaffold protein for presentation on the outer surface of the yeast, wherein the culture of yeast cells expresses a library of binding scaffold proteins, wherein the cytidine deaminase as expressed by the recombinant DNA is effective to contribute to a mutagenic process for inducing mutations in the expressed binding scaffold proteins from the yeast cell culture; such that the cytidine deaminase and the scaffold protein are expressed; (B) contacting the culture with a mutagen; and (C) selecting a subset of yeast cells that bind to a given substance more strongly than the majority of the yeast cells.

### Embodiment B2a

A method of generating a binding activity comprising: (A) cultivating a culture of yeast cells that comprise: (a) a first recombinant DNA that constitutively or inducibly expresses a cytidine deaminase with about 90% sequence identity with SEQ ID NO. 2, SEQ ID NO. 4, or a chimera between the two starting with SEQ ID NO. 2 sequence and having one transition to end in SEQ ID NO. 1 sequence; and (b) a second recombinant DNA that constitutively or inducibly expresses a binding scaffold protein for presentation on the outer surface of the yeast, wherein the culture of yeast cells expresses a library of binding scaffold proteins, wherein the cytidine deaminase as expressed by the recombinant DNA is effective to contribute to a mutagenic process for inducing mutations in the expressed binding scaffold proteins from the yeast cell culture; such that the cytidine deaminase and the scaffold protein are expressed; (B) contacting the culture with a mutagen; and (C) selecting a subset of yeast cells that bind to a given substance more strongly than the majority of the yeast cells.

### Embodiment B3

A yeast cell culture comprising: yeast cells comprising: (A) a recombinant DNA that constitutively or inducibly expresses a cytidine deaminase comprising sequence with about 90% sequence identity or more with a cytidine deaminase domain of (i) SEQ ID NO. 2 or SEQ ID NO. 4, or (ii) a chimera between the two starting with SEQ ID NO. 3 or SEQ ID NO. 4 sequence and having one transition to end in SEQ ID NO. 1 or SEQ ID NO. 2 sequence, or (iii) a chimera between the two starting with SEQ ID NO. 1 or SEQ ID NO. 2 sequence and having one transition to end in SEQ ID NO. 3 or SEQ ID NO. 4 sequence; and (B) a second recombinant DNA that constitutively or inducibly expresses a binding scaffold protein for presentation on the outer surface of the yeast, wherein the culture of yeast cells expresses a library of binding scaffold proteins, wherein the cytidine deaminase as expressed by the first recombinant DNA is effective to contribute to a mutagenic process for inducing mutations in the binding scaffold protein of the yeast cell.

### Embodiment B3a

A yeast cell culture comprising: yeast cells comprising: (A) a recombinant DNA that constitutively or inducibly expresses a cytidine deaminase comprising sequence with about 90% sequence identity or more with SEQ ID NO. 2, SEQ ID NO. 4, or a chimera between the two starting with SEQ ID NO. 3 or SEQ ID NO. 4 sequence and having one transition to end in SEQ ID NO. 1 or SEQ ID NO. 2 sequence; and (B) a second recombinant DNA that constitutively or inducibly expresses a binding scaffold protein for presentation on the outer surface of the yeast, wherein the culture of yeast cells expresses a library of binding scaffold proteins, wherein the cytidine deaminase as expressed by the first recombinant DNA is effective to contribute to a mutagenic process for inducing mutations in the binding scaffold protein of the yeast cell.

### Embodiment B4

The yeast cell, method or cell culture of a “B” Embodiment, wherein the percent identity is about 95% or higher.

### Embodiment B5

The yeast cell, method or cell culture of a “B” Embodiment, wherein the recited percent identity is to SEQ ID NO. 2.

### Embodiment B6

The yeast cell, method or cell culture of a “B” Embodiment, wherein the cytidine deaminase comprises a fused to a DNA binding domain, and wherein the second recombinant DNA comprises the cognate DNA recognition sequence to which the binding domain binds, the recognition sequence vicinal to the DNA sequence encoding the binding scaffold protein.

### Embodiment B7

The yeast cell, method or cell culture of a “B” Embodiment, wherein the DNA binding domain comprises the binding domain of estrogen receptor or GAL4 transcription factor.

### Embodiment B8

The yeast cell, method or cell culture of a “B” Embodiment, wherein the binding scaffold protein is an immunoglobulin heavy chain variable region, a light chain variable region, combinations of light and heavy chain regions, Anticalins, fibronectin type III domain, Designed Ankyrin Repeat Protein or Centyrin.

### Embodiment B9

The yeast cell, method or cell culture of a “B” Embodiment, wherein the first or second recombinant DNAs express the recited deaminase or binding scaffold protein utilizing a galactose-inducible promoter, a thermally inducible, or a copper-inducible promoter.

### Embodiment B10

The yeast cell, method or cell culture of a “B” Embodiment, wherein the first or second recombinant DNAs express the recited deaminase or binding scaffold protein utilizing a galactose-inducible promoter GAL1, a galactose-inducible promoter GAL10, a heat-shock inducible promoter HSP70, or a copper-inducible CUP1 promoter.

### Embodiment B11

The yeast cell, method or cell culture of a “B” Embodiment, wherein the yeast cell is diploid or polyploid.

### Embodiment B12

The yeast cell, method or cell culture of a “B” Embodiment, wherein the yeast cell has a deleted or inactivated HAM1 gene.

### Embodiment B13

The yeast cell, method or cell culture of a “B” Embodiment, wherein the yeast cell has a deleted or inactivated Uracil-DNA glycosylase encoding UNG1 gene.

### Embodiment B14

The method of one of a “B” Embodiment, wherein the method further comprising inducing the expression of the cytidine deaminase or library.

### Embodiment B15

The method of one of a “B” Embodiment, wherein the selecting comprising panning the cells over a surface having bound thereto the substance.

### Embodiment B16

The method of one of a “B” Embodiment, wherein the selecting further comprising selecting by cell sorting cells that more strongly bind the substance linked to a color marker.

### Embodiment B17

The method of one of a “B” Embodiment, wherein the mutagen is a nucleic acid base analog.

### Embodiment B18

The method of one of a “B” Embodiment, wherein the mutagen is a purine or pyrimidine base analogs

### Embodiment B19

The method of one of a “B” Embodiment, wherein the mutagen is 6-N-hydroxylaminopurine.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

### CITATIONS

BEERLI, R. R., M. BAUER, R. B. BUSER, M. GWERDER, S. MUNTWILER et al., 2008 Isolation of human monoclonal antibodies by mammalian cell display. Proc Natl Acad Sci USA 105: 14336-14341.
BODER, E. T., and K. D. WITTRUP, 1997 Yeast surface display for screening combinatorial polypeptide libraries. Nat Biotechnol 15: 553-557.
BODER, E. T., and K. D. WITTRUP, 2000 Yeast surface display for directed evolution of protein expression, affinity, and stability. Methods Enzymol 328: 430-444.
BURGERS, P. M., 1998 Eukaryotic DNA polymerases in DNA replication and DNA repair. Chromosoma 107: 218-227.
CHAO, G., W. L. LAU, B. J. HACKEL, S. L. SAZINSKY, S. M. LIPPOW et al., 2006 Isolating and engineering human antibodies using yeast surface display. Nat Protoc 1: 755-768.
CONRATH, K. E., M. LAUWEREYS, M. GALLENI, A. MATAGNE, J. M. FRERE et al., 2001 Beta-lactamase inhibitors derived from single-domain antibody fragments elicited in the camelidae. Antimicrob Agents Chemother 45: 2807-2812.
DRAKE, J. W., B. CHARLESWORTH, D. CHARLESWORTH and J. F. CROW, 1998 Rates of spontaneous mutation. Genetics 148: 1667-1686.
DROTSCHMANN, K., A. B. CLARK, H. T. TRAN, M. A. RESNICK, D. A. GORDENIN et al., 1999 Mutator phenotypes of yeast strains heterozygous for mutations in the MSH2 gene. Proc Natl Acad Sci USA 96: 2970-2975.
DUNCAN, B. K., and J. H. MILLER, 1980 Mutagenic deamination of cytosine residues in DNA. Nature 287: 560-561.
ECKER, D. M., S. D. JONES and H. L. LEVINE, 2015 The therapeutic monoclonal antibody market. MAbs 7: 9-14.
ENDO, K., Y. TAGO, Y. DAIGAKU and K. YAMAMOTO, 2007 Error-free RAD52 pathway and error-prone REV3 pathway determines spontaneous mutagenesis in Saccharomyces cerevisiae. Genes Genet Syst 82: 35-42.
HANES, J., and A. PLUCKTHUN, 1997 In vitro selection and evolution of functional proteins by using ribosome display. Proc Natl Acad Sci USA 94: 4937-4942.
HARRIS, R. S., S. K. PETERSEN-MAHRT and M. S. NEUBERGER, 2002 RNA editing enzyme APOBEC1 and some of its homologs can act as DNA mutators. Mol Cell 10: 1247-1253.
HAWKINS, R. E., S. J. RUSSELL and G. WINTER, 1992 Selection of phage antibodies by binding affinity. Mimicking affinity maturation. J Mol Biol 226: 889-896.
HE, M., and M. J. TAUSSIG, 1997 Antibody-ribosome-mRNA (ARM) complexes as efficient selection particles for in vitro display and evolution of antibody combining sites. Nucleic Acids Res 25: 5132-5134.
HOUSE, N. C., M. R. KOCH and C. H. FREUDENREICH, 2014 Chromatin modifications and DNA repair: beyond double-strand breaks. Front Genet 5: 296.
JIN, Y. H., A. B. CLARK, R. J. SLEBOS, H. AL-REFAI, J. A. TAYLOR et al., 2003 Cadmium is a mutagen that acts by inhibiting mismatch repair. Nat Genet 34: 326-329.
KATO, L., A. STANLIE, N. A. BEGUM, M. KOBAYASHI, M. AIDA et al., 2012 An evolutionary view of the mechanism for immune and genome diversity. J Immunol 188: 3559-3566.
KJAERULFF, S., and M. R. JENSEN, 2005 Comparison of different signal peptides for secretion of heterologous proteins in fission yeast. Biochem Biophys Res Commun 336: 974-982.
KOLODNER, R. D., and G. T. MARSISCHKY, 1999 Eukaryotic DNA mismatch repair. Curr Opin Genet Dev 9: 89-96.
KRAEBER-BODERE, F., C. BODET-MILIN, C. ROUSSEAU, T. EUGENE, A. PALLARDY et al., 2014 Radioimmunoconjugates for the treatment of cancer. Semin Oncol 41: 613-622.
LADA, A. G., C. F. KRICK, S. G. KOZMIN, V. I. MAYOROV, T. S. KARPOVA et al., 2011 Mutator effects and mutation signatures of editing deaminases produced in bacteria and yeast. Biochemistry (Mosc) 76: 131-146.
LANG, G. I., and A. W. MURRAY, 2008 Estimating the per-base-pair mutation rate in the yeast Saccharomyces cerevisiae. Genetics 178: 67-82.
MARTI, T. M., C. KUNZ and O. FLECK, 2002 DNA mismatch repair and mutation avoidance pathways. J Cell Physiol 191: 28-41.
MAYOROV, V. I., I. B. ROGOZIN, L. R. ADKISON, C. FRAHM, T. A. KUNKEL et al., 2005a Expression of human AID in yeast induces mutations in context similar to the context of somatic hypermutation at G-C pairs in immunoglobulin genes. BMC Immunol 6: 10.
MAYOROV, V. I., I. B. ROGOZIN, L. R. ADKISON, C. FRAHM, T. A. KUNKEL et al., 2005b Expression of human AID in yeast induces mutations in context similar to the context of somatic hypermutation at G-C pairs in immunoglobulin genes. BMC Immunol 6: 10.
MONTELONE, B. A., L. A. GILBERTSON, R. NASSAR, C. GIROUX and R. E. MALONE, 1992 Analysis of the spectrum of mutations induced by the rad3-102 mutator allele of yeast. Mutat Res 267: 55-66.
MONTELONE, B. A., S. PRAKASH and L. PRAKASH, 1981 Hyper-recombination and mutator effects of the mms9-1, mms13-1, and mms21-1 mutations in Saccharomyces cerevisiae. Curr Genet 4: 223-232.
MORRISON, A., J. B. BELL, T. A. KUNKEL and A. SUGINO, 1991 Eukaryotic DNA polymerase amino acid sequence required for 3′----5′ exonuclease activity. Proc Natl Acad Sci USA 88: 9473-9477.
MURAMATSU, M., K. KINOSHITA, S. FAGARASAN, S. YAMADA, Y. SHINKAI et al., 2000 Class switch recombination and hypermutation require activation-induced cytidine deaminase (AID), a potential RNA editing enzyme. Cell 102: 553-563.
MURAMATSU, M., V. S. SANKARANAND, S. ANANT, M. SUGAI, K. KINOSHITA et al., 1999 Specific expression of activation-induced cytidine deaminase (AID), a novel member of the RNA-editing deaminase family in germinal center B cells. J Biol Chem 274: 18470-18476.
NISHINO, T., and K. MORIKAWA, 2002 Structure and function of nucleases in DNA repair: shape, grip and blade of the DNA scissors. Oncogene 21: 9022-9032.
NOSKOV, V. N., K. STAAK, P. V. SHCHERBAKOVA, S. G. KOZMIN, K. NEGISHI et al., 1996 HAM1, the gene controlling 6-N-hydroxylaminopurine sensitivity and mutagenesis in the yeast Saccharomyces cerevisiae. Yeast 12: 17-29.
PANCER, Z., C. T. AMEMIYA, G. R. EHRHARDT, J. CEITLIN, G. L. GARTLAND et al., 2004 Somatic diversification of variable lymphocyte receptors in the agnathan sea lamprey. Nature 430: 174-180.
PETERSEN-MAHRT, S. K., R. S. HARRIS and M. S. NEUBERGER, 2002 AID mutates E. coli suggesting a DNA deamination mechanism for antibody diversification. Nature 418: 99-103.
REIERSEN, H., I. LOBERSLI, G. A. LOSET, E. HVATTUM, B. SIMONSEN et al., 2005 Covalent antibody display—an in vitro antibody-DNA library selection system. Nucleic Acids Res 33: e10.
ROGOZIN, I. B., L. M. IYER, L. LIANG, G. V. GLAZKO, V. G. LISTON et al., 2007 Evolution and diversification of lamprey antigen receptors: evidence for involvement of an AID-APOBEC family cytosine deaminase. Nat Immunol 8: 647-656.
SHCHERBAKOVA, P. V., and Y. I. PAVLOV, 1993 Mutagenic specificity of the base analog 6-N-hydroxylaminopurine in the URA3 gene of the yeast Saccharomyces cerevisiae. Mutagenesis 8: 417-421.
SIKORSKI, R. S., and P. HIETER, 1989 A system of shuttle vectors and yeast host strains designed for efficient manipulation of DNA in Saccharomyces cerevisiae. Genetics 122: 19-27.
STUDIER, F. W., 2005 Protein production by auto-induction in high density shaking cultures. Protein Expr Purif 41: 207-234.
TRAN, H. T., J. D. KEEN, M. KRICKER, M. A. RESNICK and D. A. GORDENIN, 1997 Hypermutability of homonucleotide runs in mismatch repair and DNA polymerase proofreading yeast mutants. Mol Cell Biol 17: 2859-2865.
VU, K. B., M. A. GHAHROUDI, L. WYNS and S. MUYLDERMANS, 1997 Comparison of llama VH sequences from conventional and heavy chain antibodies. Mol Immunol 34: 1121-1131.

All ranges recited herein include ranges therebetween, and can be inclusive or exclusive of the endpoints. Optional included ranges are from integer values therebetween (or inclusive of one original endpoint), at the order of magnitude recited or the next smaller order of magnitude. For example, if the lower range value is 0.2, optional included endpoints can be 0.3, 0.4, . . . 1.1, 1.2, and the like, as well as 1, 2, 3 and the like; if the higher range is 8, optional included endpoints can be 7, 6, and the like, as well as 7.9, 7.8, and the like. One-sided boundaries, such as 3 or more, similarly include consistent boundaries (or ranges) starting at integer values at the recited order of magnitude or one lower. For example, 3 or more includes 4 or more, or 3.1 or more. If there are two ranges mentioned, such as about 1 to 10 and about 2 to 5, those of skill will recognize that the implied ranges of 1 to 5 and 2 to 10 are within the invention.

This invention described herein is of a diversification system and methods of forming the same. Although some embodiments have been discussed above, other implementations and applications are also within the scope of the following claims. Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

Publications and references, including but not limited to patents and patent applications, cited in this specification are herein incorporated by reference in their entirety in the entire portion cited as if each individual publication or reference were specifically and individually indicated to be incorporated by reference herein as being fully set forth. Any patent application to which this application claims priority is also incorporated by reference herein in the manner described above for publications and references.

What is claimed is:

1. A yeast cell comprising:
(A) a recombinant DNA that constitutively or inducibly expresses a cytidine deaminase comprising sequence with about 90% sequence identity or more with a cytidine deaminase domain of (i) SEQ ID NO. 2 or SEQ ID NO. 4, or (ii) a chimera between the two starting with SEQ ID NO. 3 or SEQ ID NO. 4 sequence and having one transition to end in SEQ ID NO. 1 or SEQ ID NO. 2 sequence, or (iii) a chimera between the two starting with SEQ ID NO. 1 or SEQ ID NO. 2 sequence and having one transition to end in SEQ ID NO. 3 or SEQ ID NO. 4 sequence; and
(B) a second recombinant DNA that constitutively or inducibly expresses a binding scaffold protein for presentation on the outer surface of the yeast,
wherein the cytidine deaminase as expressed by the first recombinant DNA is effective to contribute to a mutagenic process for inducing mutations in the binding scaffold protein of the yeast cell.

2. The yeast cell of claim 1, wherein the percent identity is about 95% or higher.

3. The yeast cell of claim 1, wherein the recited percent identity is to SEQ ID NO. 2.

4. The yeast cell of claim 1, wherein the cytidine deaminase comprises a fused to a DNA binding domain, and wherein the second recombinant DNA comprises the cognate DNA recognition sequence to which the binding domain binds, the recognition sequence vicinal to the DNA sequence encoding the binding scaffold protein.

5. The yeast cell of claim 4, wherein the DNA binding domain comprises the binding domain of estrogen receptor or GAL4 transcription factor.

6. The yeast cell of claim 1, wherein the binding scaffold protein is an immunoglobulin heavy chain variable region, a light chain variable region, combinations of light and heavy chain regions, Anticalins, fibronectin type III domain, Designed Ankyrin Repeat Protein or Centyrin.

7. The yeast cell of claim 1, wherein the first or second recombinant DNAs express the recited deaminase or binding scaffold protein utilizing a galactose-inducible promoter, a thermally inducible, or a copper-inducible promoter.

8. The yeast cell of claim 1, wherein the first or second recombinant DNAs express the recited deaminase or binding scaffold protein utilizing a galactose-inducible promoter GAL1, a galactose-inducible promoter GAL10, a heat-shock inducible promoter HSP70, or a copper-inducible CUP1 promoter.

9. The yeast cell of claim 1, wherein the yeast cell is diploid or polyploid.

10. The yeast cell of claim 1, wherein the yeast cell has a deleted or inactivated HAM1 gene.

11. The yeast cell of claim 1, wherein the yeast cell has a deleted or inactivated Uracil-DNA glycosylase encoding UNG1 gene.

12. A method of generating a binding activity comprising:
(A) cultivating a culture of yeast cells that comprise:
(a) a first recombinant DNA that constitutively or inducibly expresses a cytidine deaminase with about 90% sequence identity with a cytidine deaminase domain of (i) SEQ ID NO. 2 or SEQ ID NO. 4, or (ii) a chimera between the two starting with SEQ ID NO. 3 or SEQ ID NO. 4 sequence and having one transition to end in SEQ ID NO. 1 or SEQ ID NO. 2 sequence, or (iii) a chimera between the two starting with SEQ ID NO. 1 or SEQ ID NO. 2 sequence and having one transition to end in SEQ ID NO. 3 or SEQ ID NO. 4 sequence; and
(b) a second recombinant DNA that constitutively or inducibly expresses a binding scaffold protein for presentation on the outer surface of the yeast, wherein the culture of yeast cells expresses a library of binding scaffold proteins, wherein the cytidine deaminase as expressed by the recombinant DNA is effective to contribute to a mutagenic process for inducing mutations in the expressed binding scaffold proteins from the yeast cell culture;
such that the cytidine deaminase and the scaffold protein are expressed;
(B) contacting the culture with a mutagen; and
(C) selecting a subset of yeast cells that bind to a given substance more strongly than the majority of the yeast cells.

13. The method of claim 12, wherein the percent identity is about 95% or higher.

14. The method of claim 12, wherein the recited percent identity is to SEQ ID NO. 2.

15. The method of claim 12, wherein the method further comprising inducing the expression of the cytidine deaminase or library.

16. The method of claim 12, wherein the selecting comprising panning the cells over a surface having bound thereto the substance.

17. The method of claim 12, wherein the selecting further comprising selecting by cell sorting cells that more strongly bind the substance linked to a color marker.

18. The method of claim 12, wherein the mutagen is a nucleic acid base analog.

19. The method of claim 12, wherein the mutagen is a purine or pyrimidine base analogs

20. The method of claim 12, wherein the mutagen is 6-N-hydroxylaminopurine.
